(19) United States
(12) Patent Application Publication
(10) Pub. No.: US 2018/0371542 A1
(43) Pub. Date: Dec. 27, 2018

(54) OPIOID RECEPTOR BLOCKADE IN TREATING ALCOHOL USE DISORDERS

(71) Applicant: MUSC Foundation for Research Development, Charleston, SC (US)

(72) Inventor: Raymond F. Anton, Charleston (US)

(73) Assignee: MUSC Foundation for Research Development, Charleston, SC (US)

(21) Appl. No.: 16/019,091

(22) Filed: Jun. 26, 2018

Related U.S. Application Data
(60) Provisional application No. 62/525,123, filed on Jun. 26, 2017.

(51) Int. Cl. C12Q 1/6876; A61K 31/485; A61P 25/32

(52) U.S. Cl. CPC C12Q 1/6876; A61K 31/485; C12Q 2600/106; C12Q 2600/156; A61P 25/32

(57) ABSTRACT

Provided are methods for treating alcohol use disorders using opioid receptor antagonists. In some embodiments, the presently disclosed methods include assaying nucleic acid from a subject regarding the subject's genotype with respect to the COMT and OPRM1 genes and administering or not administering an opioid receptor antagonist to the subject on the basis therefore. Also provided are methods for detecting susceptibility to an opioid receptor antagonist therapy for disorders associated with opioid receptor activity and methods for identifying and treating human subjects having susceptibility to opioid receptor antagonist therapies for disorders associated with opioid receptor activity.

### CROSS-REFERENCE TO RELATED APPLICATION

This application claims the benefit of U.S. Provisional Patent Application Ser. No. 62/525,123, filed Jun. 26, 2017, the disclosure of which is incorporated herein by reference in its entirety.

### GRANT STATEMENT

This invention was made with government support under R01AA017633 and P50 AA010761, and K05AA017435 awarded by the National Institute on Alcohol Abuse and Alcoholism. The government has certain rights in the invention.

### TECHNICAL FIELD

The presently disclosed subject matter relates to treating disorders associated with opioid receptor activity, particularly to methods for treating disorders associated with opioid receptor activity including but not limited to alcohol use disorders using opioid receptor antagonists, and also to methods for detecting susceptibility to opioid receptor antagonist therapy for disorders associated with opioid receptor activity.

### BACKGROUND

It is well documented that naltrexone is efficacious in the treatment of alcohol dependence and has been approved by the U.S. Food and Drug Administration for this indication since 1994. Nevertheless, the effect is moderate at best, and it is recognized that not all individuals with an alcohol use disorder (AUD) respond to it. Genetic differences have been suggested as one factor that might influence both response to alcohol and the ability of naltrexone to modify this response. There have been a number of animal and human clinical laboratory studies, suggesting that a single nucleotide polymorphism (SNP) in the mu opioid gene (OPRM1) (A118G) leading to a missense asparagine to aspartic acid amino acid substitution at position 40 (Asn40Asp; rs1799971) can lead to differences in alcohol effects and response to naltrexone. It has been shown that some of this enhanced alcohol response and drinking behavior in rodents engineered to have a homologous SNP is because of increased dopamine release in the nucleus accumbens, which in general is thought to be a signature of reinforcement and addiction. Of interest, naltrexone has been shown in animals to both reduce nucleus accumbens dopamine release and to reduce drinking in rodent and nonhuman primate drinking models. Also of interest, a homologous SNP to that found in humans in the mu opioid receptor gene occurring naturally in non-human primates also appears to confer both sensitivity to alcohol response and response to naltrexone in the reduction of alcohol effects and consumption. This finding parallels data in human clinical laboratory studies and clinical trials where naltrexone appears to exert a stronger effect on those individuals with the Asp40 OPRM1 (A118G) SNP. However, this finding is not universal as several reports suggest that it may not be as salient.

What is needed are new methods of treating AUD with opioid receptor antagonists (such as, for example naltrexone) that remove these inconsistencies and methods of detecting susceptibility to opioid receptor antagonist treatment.

### SUMMARY

This Summary lists several embodiments of the presently disclosed subject matter, and in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods and compositions related to the use of opioid receptor antagonists in the treatment of psychiatric, mental, and/or neurological disorders.

In some embodiments, the presently disclosed methods relate to treating psychiatric, mental, and/or neurological disorders (such as, for example, alcohol use disorder (AUD)). In some embodiments, the presently disclosed methods comprise assaying the nucleic acid from a subject for the genotype of the dopamine (DA)-catabolizing enzyme catechol-O-methyltransferase (COMT) and the genotype of the opioid mu receptor gene (OPRM1), wherein when one or two alleles encoding a methionine at amino acid residue 158 of COMT and two alleles encoding an asparagine at amino acid residue 40 of OPRM1 is detected or wherein two alleles encoding valine at amino acid residue 158 of COMT and at least one allele encoding an aspartic acid at amino acid residue 40 of OPRM1 is detected, an opioid receptor antagonist is administered to the subject; and wherein when one or two alleles encoding a methionine at amino acid residue 158 of COMT and at least one allele encoding an aspartic acid at amino acid residue 40 of OPRM1 is detected or wherein two alleles encoding valine at amino acid residue 158 of COMT and two alleles encoding an asparagine at amino acid residue 40 of OPRM1 is detected, an opioid receptor antagonist is not administered to the subject. In some embodiments, the genotype of COMT is assayed prior to administering the opioid receptor antagonist or wherein the genotype of COMT is assayed after opioid receptor antagonist therapy has commenced, and wherein when one or two alleles encoding a methionine at amino acid residue 158 of COMT and at least one allele encoding an aspartic acid at amino acid residue 40 of OPRM1 is detected or wherein two alleles encoding valine at amino acid residue 158 of COMT and two alleles encoding an asparagine at amino acid residue 40 of OPRM1 is detected, the opioid receptor antagonist therapy is discontinued. In some embodiments, the opioid receptor antagonist is a naltrexone. In some embodiments, the genotype of the COMT and OPRM1 polymorphisms are detected by a nucleic acid amplification process followed by sequencing or gel electrophoresis or direct sequencing.

In some embodiments, the presently disclosed methods further comprise assaying the nucleic acid from a subject for the genotype of the variable number tandem repeats (VNTR) polymorphism in the dopamine transporter gene DAT1/SLC6A3, wherein when one or two alleles for 9 tandem repeats and an asparagine at amino acid residue 40 of OPRM1 is detected or wherein two alleles for 10 tandem repeats and an aspartic acid at amino acid residue 40 of OPRM1 is detected, an opioid receptor antagonist is administered to the subject; and wherein when one or two alleles for 9 tandem repeats and an aspartic acid at amino acid residue 40 of OPRM1 is detected or wherein two alleles for 10 tandem repeats and an asparagine at amino acid residue 40 of OPRM1 is detected, an opioid receptor antagonist is not administered to the subject.

In some embodiments, the presently disclosed subject matter also provides methods for detecting susceptibility to an opioid receptor antagonist therapy for alcohol use disorder. In some embodiments, the presently disclosed methods comprise obtaining a tissue sample from a subject, assaying nucleic acid from the tissue sample from the subject for the genotype encoding the amino acid substitution at residue 158 of the DA-catabolizing enzyme catechol-O-methyltransferase (COMT) and the genotype encoding an amino acid substitution at residue 40 of the opioid mu receptor gene (OPRM1), wherein when one or two alleles encode for a methionine at amino acid residue 158 of COMT and a genome that encodes for an asparagine at amino acid residue 40 of OPRM1 or wherein two alleles encoding valine at amino acid residue 158 of COMT and the genome encodes for an aspartic acid at amino acid residue 40 of OPRM1 indicates that the subject is susceptible to dopamine modulator therapy.

In some embodiments of the presently disclosed methods, the opioid receptor antagonist is a naltrexone.

In some embodiments of the presently disclosed methods, the genotype of the OPRM1 and COMT polymorphisms are detected by a nucleic acid amplification process followed by sequencing or gel electrophoresis or direct sequencing.

In some embodiments, the presently disclosed methods further comprise assaying the nucleic acid from a subject for the genotype of the variable number tandem repeats (VNTR) polymorphism in the dopamine transporter gene DAT1/SLC6A3.

The presently disclosed subject matter also provide in some embodiments methods for treating subjects with disorders associated with opioid receptor activity. In some embodiments, the methods comprising performing or having performed one or more genotyping assays on nucleic acids isolated from the subject to determine the subject's genotype with respect to a first gene and a second gene, wherein the first gene is an opioid mu receptor (OPRM1) gene and the second gene is selected from the group consisting of a dopamine (DA)-catabolizing enzyme catechol-O-methyltransferase (COMT) gene and a dopamine transporter DAT1/SLC6A3 gene; and administering an opioid receptor antagonist therapy to the subject when the subject's genotype has two alleles encoding an asparagine at an amino acid position corresponding to amino acid residue 40 the of OPRM1 gene product of SEQ ID NO: 12 and has at least one allele encoding a methionine at an amino acid position corresponding to amino acid residue 158 of the COMT gene product of SEQ ID NO: 8 or at least one allele encoding a dopamine transporter DAT1/SLC6A3 9-repeat variable number tandem repeat (VNTR); or the subject's genotype has at least one allele encoding an aspartic acid at an amino acid position corresponding to amino acid residue 40 the of OPRM1 gene product of SEQ ID NO: 12 and has two alleles encoding a valine at an amino acid position corresponding to amino acid residue 158 of the COMT gene product of SEQ ID NO: 8 or two alleles encoding a dopamine transporter DAT1/SLC6A3 10-repeat variable number tandem repeat (VNTR). In some embodiments, the disorder associated with opioid receptor activity is an alcohol use disorder (AUD). In some embodiments, at least one of the one or more genotyping assays is performed prior to administering the opioid receptor antagonist therapy to the subject. In some embodiments, at least one of the one or more genotyping assays are performed after administering the opioid receptor antagonist therapy to the subject, and further wherein the opioid receptor antagonist therapy is discontinued if the subject has at least one allele encoding an aspartic acid at an amino acid position corresponding to amino acid residue 40 of the OPRM1 gene product of SEQ ID NO: 12 and at least one allele encoding a methionine at an amino acid position corresponding to amino acid residue 158 of the COMT gene product of SEQ ID NO: 8 or at least one allele for a DAT1/SLC6A3 9-repeat variable number tandem repeat (VNTR); or the subject is homozygous for an asparagine at an amino acid position corresponding to amino acid residue 40 the of OPRM1 gene product of SEQ ID NO: 12 and is homozygous for a valine at an amino acid position corresponding to amino acid residue 158 of the COMT gene product of SEQ ID NO: 8 or is homozygous for a DAT1/SLC6A3 10-repeat variable number tandem repeat (VNTR). In some embodiments, the opioid receptor antagonist therapy comprises administering an effective amount of naltrexone to the subject. In some embodiments, at least one of the one or more genotyping assays comprises a nucleic acid amplification process followed by sequencing or gel electrophoresis of an amplification product produced thereby.

Thus, it is an object of the presently disclosed subject matter to provide methods for treating psychiatric, mental, and/or neurological disorders associated with opioid receptor activity (such as, for example, alcohol use disorder (AUD)) using opioid receptor antagonists.

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

A more complete understanding of the presently disclosed subject matter can be obtained by reference to the accompanying Figures, when considered in conjunction with the subsequent Detailed Description. The embodiments illustrated in the Figures are intended to be exemplary only, and should not be construed as limiting the presently disclosed subject matter to the illustrated embodiments. The accompanying Figures, which are incorporated in and constitute a part of this disclosure, thus illustrate several embodiments and together with the description taken in its entirety are illustrative of the disclosed compositions and methods.

FIG. 1 is a flow chart showing consolidated standards of reporting Clinical Trials Diagram. Legend: Those with valid data had more than one week of drinking data reported post-randomization.

FIGS. 2A and 2B are a series of graphs showing the percent heavy drinking days (% HDD) over the 4 study months. % HDD was analyzed in mixed model of medication (naltrexone (triangles) vs. placebo (open circles))×OPRM1 allele×COMT (COMT158 Met Carrier vs. Val/Val homozygotes)×time (study weeks). FIG. 2A shows the results for OPRM1 Asn/Asn homozygotes, with the top panel being COMT158 Met Carriers and the bottom panel being COMT158 Val/Val homozygotes. FIG. 2B shows the results for OPRM1 Asp carriers, with the top panel being COMT158 Met Carriers and the bottom panel being COMT158 Val/Val homozygotes. Statistically for Medication×OPRM1×COMT is (F=4.25, p=0.041).

FIG. 3 is a bar graph showing the percent heavy drinking days (% HDD) over the 4 months of the study. % HDD was analyzed as univariate mixed model of medication (naltrexone, black squares vs. placebo, gray squares)×OPRM1 allele (at least one Asp40 allele, right two pairs of bars vs. Asn/Asn homozygotes, left two pairs of bars)×COMT (at least one COMT Met158 allele, first and third bar pairs vs. Val/Val homozygotes, second and fourth bar pairs)×time (4 months). Values for treatment response are given for each gene pair by medication group. Significant treatment response is obtained only for those who are OPRM1 Asn (A allele) homozygotes who are also COMT 158 Met (G allele) carriers (p=0.03) and for those who are OPRM1 Asp40 (G allele) carriers who are COMT 158 Val/Val (A/A allele) homozygotes (p=0.05). Statistically, p=0.065 for Medication×OPRM1×COMT×F=3.47.

FIGS. 4A and 4B are a series of graphs showing the percent heavy drinking days (% HDD) over the 4 study months. % HDD was analyzed in mixed model of medication (naltrexone (triangles) vs. placebo (open circles))×OPRM1 allele×DAT1 VNTR allele (9R carriers vs. 10R homozygotes)×time (study weeks). FIG. 4A shows the results for OPRM1 Asn/Asn homozygotes, with the top panel being DAT1 VNTR 9R carriers and the bottom panel being DAT1 VNTR 10R homozygotes. FIG. 4B shows the results for OPRM1 Asp carriers, with the top panel being DAT1 VNTR 9R carriers and the bottom panel being DAT1 VNTR 10R homozygotes. Medication×OPRM1×DAT by time (F=3.63 p=0.015)

FIG. 5 is a bar graph showing the number of Drinks Per Drinking Day (DPDD) in the end (month 4) of the trial. % HDD was analyzed in mixed model of medication (naltrexone, black squares vs. placebo, gray squares)×OPRM1 allele (at least one Asp40 allele, third and fourth pairs of bars vs. Asn/Asn homozygotes, first and second pairs of bars)×DAT1 VNTR genotype (at least one 9R allele, first and third bar pairs vs. 10R homozygotes, second and fourth bar pairs)×time (4 months). Statistically, p=0.017 for Medication×OPRM1×DAT1 VNTR×F=5.98.

### BRIEF DESCRIPTION OF THE SEQUENCE LISTING

SEQ ID NOs: 1 and 2 are the nucleotide sequences of an exemplary oligonucleotide pair that together can be employed for determining the number of DAT1/SLC6A3 VNTR repeats in a nucleic acid sample.

SEQ ID NO: 3 is the nucleotide sequence of an exemplary oligonucleotide that can be employed with SEQ ID NO: 2 for determining the number of DAT1/SLC6A3 VNTR repeats in a nucleic acid sample.

SEQ ID NO: 4 is the sequence of the 40-base-pair variable number tandem repeat (VNTR) polymorphism. It corresponds to the insertion/deletion (indel) variation single nucleotide polymorphism (SNP) rs28363170 of the human DAT1/SLC6A3 VNTR, where the 9-repeat (9R) allele has nine consecutive repeats of SEQ ID NO: 4 and the 10-repeated (10R) allele has ten consecutive repeats of SEQ ID NO: 4.

SEQ ID NO: 5 is the nucleotide sequence of the single nucleotide polymorphism (SNP) rs1799971 of the human OPRM1 gene. The polymorphism is located at nucleotide 26 of SEQ ID NO: 5 (this position corresponds to nucleotide position 561 of SEQ ID NO: 11), wherein the nucleotide at this position is in some embodiments an adenine and is in some embodiments a guanine. With respect to the exemplary OPRM1 amino acid sequence of SEQ ID NO: 12, amino acid position 40 is shown as an asparagine residue as SEQ ID NO: 11 shows position 561 to be an adenine, but when the nucleotide at position 561 of SEQ ID NO: 11 is a guanine, the amino acid at the amino acid position that corresponds to residue number 40 of SEQ ID NO: 12 would be an aspartic acid.

SEQ ID NO: 6 is the nucleotide sequence of the single nucleotide polymorphism (SNP) rs4680 of the human COMT gene. The polymorphism is located at nucleotide 26 of SEQ ID NO: 6 (corresponds to nucleotide 721 of SEQ ID NO: 7), wherein the nucleotide at this position is in some embodiments a guanine and is in some embodiments an adenine. With respect to the exemplary COMT amino acid sequence of SEQ ID NO: 8, amino acid position 158 is shown as a valine residue as SEQ ID NO: 7 shows position 721 to be a guanine, but when the nucleotide at position 721 of SEQ ID NO: 7 is an adenine, the amino acid at the amino acid position that corresponds to residue number 158 of SEQ ID NO: 8 is a methionine.

SEQ ID NOs: 7 and 8 are nucleotide and amino acid sequences, respectively, of exemplary human COMT gene products. The nucleotide sequence corresponds to Accession No. NM_000754.3 in the GENBANK® biosequence database, and the amino acid sequence corresponds to Accession No. NP_000745.1 in the GENBANK® biosequence database.

SEQ ID NOs: 9 and 10 are nucleotide and amino acid sequences, respectively, of exemplary human DAT1/SLC6A3 gene products. The nucleotide sequence corresponds to Accession No. NM_001044.4 in the GENBANK® biosequence database, and the amino acid sequence corresponds to Accession No. NP_001035.1 in the GENBANK® biosequence database.

SEQ ID NOs: 11 and 12 are nucleotide and amino acid sequences, respectively, of exemplary human OPRM1 gene products. The nucleotide sequence corresponds to Accession No. NM_000914.4 in the GENBANK® biosequence database, and the amino acid sequence corresponds to Accession No. NP_000905.3 in the GENBANK® biosequence database. There are numerous different transcription and/or splice variants of the human OPRM1 gene, all of which are encompassed within the presently disclosed subject matter. As such, SEQ ID NOs: 11 and 12 are intended to be exemplary only and not limiting.

As is known in the art, in some embodiments multiple gene products can be generated from a particular genetic locus, for example by alternative transcriptional initiation sites, alternative splicing, etc. It is understood that the GENBANK® Accession Nos. presented herein are meant to be exemplary only, and other gene products for which the nucleotide and/or amino acid sequences are not explicitly disclosed herein are also intended to be encompassed by the names of the corresponding genes. Thus, for example, transcript variants of the sequences in the Sequence Listing are also included with the definitions of the genes described herein, as are the amino acid variants encoded thereby.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Headings are included herein for reference and to aid in locating certain sections. These headings are not intended to limit the scope of the concepts described therein under, and these concepts can have applicability in other sections throughout the entire description.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

As used in the specification and the appended claims, the singular forms “a,” “an”, and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, in some embodiments the range includes from the one particular value and/or to the other particular value. Similarly, when values are expressed as approximations, such as but not limited to by use of the antecedent “about,” it will be understood that the particular value forms an exemplary non-limiting embodiment. It will be further understood that the endpoints of each of the ranges are significant both in relation to the other endpoint, and independently of the other endpoint.

It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, “less than or equal to 10” as well as “greater than or equal to 10” are also disclosed. It is also understood that throughout the disclosure, data are provided in a number of different formats, and that these data represent in some embodiments endpoints and starting points, and ranges for any combination of the data points. By way of example and not limitation, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Unless otherwise indicated, all numbers expressing quantities of components, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

In this disclosure and in the claims which follow, reference will be made to a number of terms and phrases which shall be defined to have the following meanings:

“Optional” or “optionally” means that the subsequently described event or circumstance may or may not occur, and that the description includes instances where said event or circumstance occurs and instances where it does not.

“Probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

“Primers” are a subset of probes which are capable of supporting some type of enzymatic manipulation and which can hybridize with a target nucleic acid such that the enzymatic manipulation can occur. A primer can be made from any combination of nucleotides and/or nucleotide derivatives and/or analogs available in the art which do not interfere with the enzymatic manipulation.

Throughout this disclosure, various publications are referenced. The disclosures of these publications are hereby incorporated by reference into this application in their entireties in order to more fully describe the state of the art to which this pertains. The references disclosed are also individually and specifically incorporated by reference herein for the material contained in them that is discussed in the sentence in which the reference is relied upon.

II. Disorders Associated with Opioid Receptor Activities

In some embodiments, the presently disclosed subject matter provides methods for treating subjects with disorders associated with opioid receptor activity, detecting susceptibility of subject to treatment with an opioid receptor antagonist for disorders associated with opioid receptor activity, and identifying and treating subjects (e.g., human subjects) having susceptibility to opioid receptor agonist and/or antagonist therapies for disorders associated with opioid receptor activity in the subjects.

As used herein, the phrase “disorder associated with opioid receptor activity” refers to any disease, disorder, or condition at least one symptom of which can be improved or treated by administering to a subject in need thereof an opioid receptor antagonist or an opioid receptor agonist, depending on whether the symptom results from undesirably high opioid receptor activity or undesirably low opioid receptor activity. Exemplary disorders associated with opioid receptor activity include, but are not limited to alcohol use disorders (AUD), opiate addiction and/or abuse, whether intentional or unintentional; depression, anxiety, compulsive disorders, and pain.

As used herein, the phrases “opioid antagonist” and “opioid receptor antagonist” refer to any agent that inhibits signaling through an opioid receptor either directly or indirectly. Exemplary opioid receptor antagonists include naltrexone, naloxone, nalmefene, and buprenorphine. See also Niciu & Arias, 2013. Other agents that have opioid receptor antagonist activities include, but are not limited to nalorphine, nalorphine dinicotinate, levallorphan, samidorphan, and nalodeine.

As used herein, the phrase “opioid agonist” and “opioid receptor agonist” refer to any agent that enhances or augments signaling through an opioid receptor either directly or indirectly. Exemplary opioid receptor agonists include methadone, buprenorphine, oxycodone, hydrocodone, codeine and its derivatives, pethidine, fentanyl, morphine, and hydromorphone.

As is known in the art, some antagonists and agonists have overlapping activities such that under different circumstances they can act as either antagonists or agonists. Exemplary such agents include partial agonists, competitive antagonists, inverse agonists, and mixed agonist/antagonists. As used herein, whether a given agent acts as an antagonist or an agonist can depend on the disorder for which use of the antagonist or agonist is desired.

An exemplary disorder associated with opioid receptor activity is alcohol use disorder (AUD), a chronic relapsing brain disease characterized by compulsive alcohol use, loss of control over alcohol intake, and a negative emotional state when not using. An estimated 16 million people have been diagnosed as having AUD in the United States alone. To be diagnosed with AUD, individuals must meet at least two of the criteria outlined in the Diagnostic and Statistical Manual of Mental Disorders (DSM) including amount or duration of consumption, inability to reduce or stop drinking, time spent drinking or recovering, craving, interference of drinking on work, school, or family, maintaining consumption despite problems resulting from consumption, reducing activities to place more emphasis on consumption, increased risk behavior while consuming or intoxicated, continued consumption despite feelings of depression or anxiety, increased average consumption over the past year, and presence of withdrawal symptoms.

Treatment for AUD can comprise counseling, behavioral modification, and pharmacological intervention. Currently, three drugs, Naltrexone, Acamprosate, and Disulfiram are approved for treating alcohol use disorder.

There have been reports that functional genetic differences in several dopamine system genes, including but not limited to the OPRM1 gene, alter reward-based brain mechanisms. Dopamine (DA) signaling regulates many aspects of AUD. Alcohol cues and intravenous alcohol self-administration both increase DA release in the human ventral striatum (VS). Individuals with AUD, relative to controls, display enhanced alcohol-induced but blunted amphetamine-induced VS DA release, and, unlike controls, demonstrate no association between striatal DA release and prefrontal glucose metabolism, suggesting impaired cortical modulation of VS DA signaling.

Because it has been speculated that dopamine tone/release might underlie the stimulant response to alcohol observed in heavy drinkers and early-stage alcoholics and because naltrexone reduces ventral striatal dopamine output in rodents and also reduces alcohol-induced stimulation in man, it is likely that there might be a salient interaction between the opiate and dopamine systems that might be genetically based and that can be modified by naltrexone and/or other opioid antagonists. For instance, people who are carriers of the DAT1 9-repeat VNTR might be more likely to have elevated dopamine levels in nucleus accumbens after alcohol consumption or cue-presentation and therefore be more likely to respond to naltrexone—secondary to its ability to decrease alcohol or cue-induced dopamine release. Similarly, certain alleles of the COMT rs4680 SNP (also known as Val158Met) might be more likely to have elevated dopamine levels. The COMT Met allele has been associated with reduced catechol-O-methyltransferase efficiency, likely increasing extrasynaptic DA accumulation, and also with heightened DA receptor sensitivity among AUD individuals.

In some embodiments, disclosed herein are methods of treating a psychiatric, mental, and/or neurological disorder (such as, for example, AUD) comprising assaying a nucleic acid from a subject with respect to the genotype of the dopamine (DA)-catabolizing enzyme catechol-O-methyltransferase (COMT) and the genotype of the opioid mu receptor gene (OPRM1), wherein when one or two alleles encoding a methionine at amino acid residue 158 of COMT and two copies of an allele encoding an asparagine at amino acid residue 40 of OPRM1 is detected or wherein two alleles encoding valine at amino acid residue 158 of COMT and at least one allele encoding an aspartic acid at amino acid residue 40 of OPRM1 is detected, an opioid receptor antagonist is administered to the subject; and wherein when one or two alleles encoding a methionine at amino acid residue 158 of COMT and at least one allele encoding an aspartic acid at amino acid residue 40 of OPRM1 is detected or wherein two alleles encoding valine at amino acid residue 158 of COMT and two alleles encoding an asparagine at amino acid residue 40 of OPRM1 is detected, an opioid receptor antagonist is not administered to the subject.

It is understood and herein provided that the detection genotype COMT and OPRM1 can also be used to detect susceptibility to opioid receptor antagonist therapy. Accordingly, disclosed herein are methods of detecting susceptibility to an opioid receptor antagonist therapy for alcohol use disorder comprising obtaining a tissue sample from a subject, assaying nucleic acid from the tissue sample from the subject for the genotype encoding the amino acid substitution at residue 158 of the DA-catabolizing enzyme catechol-O-methyltransferase (COMT) and the genotype encoding an amino acid substitution at residue 40 of the opioid mu receptor gene (OPRM1), wherein when one or two alleles encode for a methionine at amino acid residue 158 of COMT and a genome that encodes for an asparagine at amino acid residue 40 of OPRM1 or wherein two alleles encoding valine at amino acid residue 158 of COMT and the genome encodes for an aspartic acid at amino acid residue 40 of OPRM1 indicates that the subject is susceptible to opioid receptor antagonist therapy.

As used in the methods of treating a psychiatric, mental, and/or neurological disorder (such as, for example, alcohol use disorder (AUD)) or methods of detecting susceptibility to opioid receptor antagonist treatment disclosed herein, a opioid receptor antagonist as used in the disclosed methods can comprise any antibody, biologic, small molecule, and/or nucleic acid modulators (including, but not limited to small interfering RNAs (siRNAs), small hairpin RNAs (shRNAs), antisense molecules, zinc finger nucleases, meganucleases, TAL (TALE) nucleases, triplexes, modified triplexes). Such opioid receptor antagonists can include but are not limited to naloxone, naltrexone, nalmefene, diprenorphine, nalorphine, nalorphine dinicotinate, levallorphan, samidorphan, nalodeine, alvimopan, methylnaltrexone, naloxegol, 6β-naltrexol, axelopran, bevenopran, methylsamidorphan, and naldemedine.

In some embodiments, it is understood and herein provided that the disclosed treatment methods can be applied prior to any opioid receptor antagonist therapy and/or as a modification of an opioid receptor antagonist therapy. Thus, in some embodiments, disclosed herein are methods of treating a psychiatric, mental, and/or neurological disorder (such as, for example, alcohol use disorder (AUD)) comprising assaying the nucleic acid from a subject for the genotype of the dopamine (DA)-catabolizing enzyme catechol-O-methyltransferase (COMT) and the genotype of the opioid mu receptor gene (OPRM1), wherein the genotype of COMT is assayed prior to administering the opioid receptor antagonist. Also disclosed are methods of treating a psychiatric, mental, and/or neurological disorder (such as, for example, alcohol use disorder (AUD)), wherein the genotype of COMT is assayed after opioid receptor antagonist therapy has commenced, and wherein when one or two alleles encoding a methionine at amino acid residue 158 of COMT and at least one allele encoding an aspartic acid at amino acid residue 40 of OPRM1 is detected or wherein two alleles encoding valine at amino acid residue 158 of COMT and two alleles encoding an asparagine at amino acid residue 40 of OPRM1 is detected, the opioid receptor antagonist therapy is discontinued.

It is understood and herein provided that the disclosed methods of treating, methods of detecting the susceptibility to opioid receptor antagonist therapy and kits are not limited to alcohol use disorder, but can be used for any psychiatric, mental, and/or neurological disorder where opioid receptor modulation can have an effect on the treatment of the subject. For example, the psychiatric, mental, and/or neurological disorder can comprise schizophrenia, bipolar disorder, depression, and chemical addition (including, but not limited to cocaine addiction, opioid addiction, amphetamine (including methamphetamine) addiction, nicotine addiction, prescription drug addiction, and alcohol use disorder).

The disclosed methods of detection and treatment assay for nucleic acid polymorphisms of the COMT and OPRM1 and/or COMT, the VNTR of DAT1, and OPRM1. Such polymorphisms can be detected using any method known in the art for detection of nucleic acids.

### III. DNA Detection and Quantification

As indicated throughout, the methods disclosed herein relate to the detection of nucleic acid variation in the form of, for example, the alleles for the polymorphism encoding the amino acid at residue 158 of COMT, the allele for the polymorphism encoding the amino acid at residue 40 of OPRM1, and the allele for the number of variable number tandem repeats (VNTR) of DAT1. For these latter expression level detections, the methods comprise detecting either the abundance or presence of mRNA, or both. Alternatively, detection can be directed to the abundance or presence of DNA, for example, cDNA. Thus, disclosed herein are methods of treating a psychiatric, mental, and/or neurological disorder (such as, for example, alcohol use disorder (AUD)) comprising assaying the nucleic acid from a subject for the genotype of the dopamine (DA)-catabolizing enzyme catechol-O-methyltransferase (COMT) and the genotype of the opioid mu receptor gene (OPRM1), wherein when one or two alleles encoding a methionine at amino acid residue 158 of COMT and two alleles encoding an asparagine at amino acid residue 40 of OPRM1 is detected or wherein two alleles encoding valine at amino acid residue 158 of COMT and at least one allele encoding an aspartic acid at amino acid residue 40 of OPRM1 is detected, an opioid receptor antagonist is administered to the subject; and wherein when one or two alleles encoding a methionine at amino acid residue 158 of COMT and at least one allele encoding an aspartic acid at amino acid residue 40 of OPRM1 is detected or wherein two alleles encoding valine at amino acid residue 158 of COMT and two alleles encoding an asparagine at amino acid residue 40 of OPRM1 is detected, an opioid receptor antagonist is not administered to the subject; wherein the genotype of the COMT and OPRM1 polymorphisms (and the genotype of the variable number tandem repeats (VNTR) polymorphism in the dopamine transporter gene DAT1/SLC6A3 when included) are detected by a nucleic acid amplification process followed by sequencing or gel electrophoresis or by direct sequencing. Also disclosed herein are methods of detecting susceptibility to an opioid receptor antagonist therapy for alcohol use disorder comprising obtaining a tissue sample from a subject, assaying nucleic acid from the tissue sample from the subject for the genotype encoding the amino acid substitution at residue 158 of the DA-catabolizing enzyme catechol-O-methyltransferase (COMT) and the genotype encoding an amino acid substitution at residue 40 of the opioid mu receptor gene (OPRM1), wherein when one or two alleles encode for a methionine at amino acid residue 158 of COMT and a genome that encodes for an asparagine at amino acid residue 40 of OPRM1 or wherein two alleles encoding valine at amino acid residue 158 of COMT and the genome encodes for an aspartic acid at amino acid residue 40 of OPRM1 indicates that the subject is susceptible to dopamine modulator therapy; wherein the genotype of the COMT and OPRM1 polymorphisms (and the genotype of the variable number tandem repeats (VNTR) polymorphism in the dopamine transporter gene DAT1/SLC6A3 when included) are detected by a nucleic acid amplification process followed by sequencing or gel electrophoresis or by direct sequencing.

A number of widely used procedures exist for detecting and determining the abundance of a particular DNA in a sample. For example, the technology of PCR permits amplification and subsequent detection of minute quantities of a target nucleic acid. Details of PCR are well described in the art, including, for example, U.S. Pat. Nos. 4,683,195 to Mullis et al., 4,683,202 to Mullis, and 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. It is understood and herein contemplated that there are variant PCR methods known in the art that may also be utilized in the disclosed methods, for example, Quantitative PCR (QPCR); microarrays, real-time PCT; hot start PCR; nested PCR; allele-specific PCR; digital droplet PCR (ddPCR), digital droplet quantitative PCR (ddQPCR), and Touchdown PCR.

III.A. Microarrays

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GENECHIP® (Affymetrix, Inc which refers to its high density, oligonucleotide-based DNA arrays), and gene array.

DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

There are two variants of the DNA microarray technology, in terms of the property of arrayed DNA sequence with known identity. Type I microarrays comprise a probe cDNA (typically about 500-5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or noncovalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink-jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate.

Tissue samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art.

Methods for detecting complex formation are well known to those skilled in the art. In one embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Type II microarrays comprise an array of oligonucleotides (typically about 20 to about 80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GENECHIP® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

III.B. Real-Time PCR

Real-time PCR monitors the fluorescence emitted during the reaction as an indicator of amplicon production during each PCR cycle (i.e., in real time) as opposed to the endpoint detection. The real-time progress of the reaction can be viewed in some systems. Real-time PCR does not detect the size of the amplicon and thus does not allow the differentiation between DNA and cDNA amplification; however, it is not influenced by non-specific amplification unless SYBR Green is used. Real-time PCR quantitation eliminates post-PCR processing of PCR products. This helps to increase throughput and reduce the chances of carryover contamination. Real-time PCR also offers a wide dynamic range of up to 10⁷-fold. Dynamic range of any assay determines how much target concentration can vary and still be quantified. A wide dynamic range means that a wide range of ratios of target and normalizer can be assayed with equal sensitivity and specificity. It follows that the broader the dynamic range, the more accurate the quantitation. When combined with RT-PCR, a real-time RT-PCR reaction reduces the time needed for measuring the amount of amplicon by providing for the visualization of the amplicon as the amplification process is progressing.

The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles can indicate the detection of accumulated PCR product.

A fixed fluorescence threshold is set significantly above the baseline that can be altered by the operator. The parameter C_{T} (threshold cycle) is defined as the cycle number at which the fluorescence emission exceeds the fixed threshold.

There are three main fluorescence-monitoring systems for DNA amplification: (1) hydrolysis probes; (2) hybridizing probes; and (3) DNA-binding agents. Hydrolysis probes include TAQMAN® brand probes, molecular beacons and scorpions. They use the fluorogenic 5′ exonuclease activity of Taq polymerase to measure the amount of target sequences in cDNA samples.

TAQMAN® brand probes are oligonucleotides longer than the primers (20-30 bases long with a Tm value of 10° C. higher) that contain a fluorescent dye usually on the 5′ base, and a quenching dye (usually TAMRA) typically on the 3′ base. When irradiated, the excited fluorescent dye transfers energy to the nearby quenching dye molecule rather than fluorescing (this is called FRET=Förster or fluorescence resonance energy transfer). Thus, the close proximity of the reporter and quencher prevents emission of any fluorescence while the probe is intact. TAQMAN® brand probes are designed to anneal to an internal region of a PCR product. When the polymerase replicates a template on which a TAQMAN® brand probe is bound, its 5′ exonuclease activity cleaves the probe. This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TAQMAN® brand assay uses universal thermal cycling parameters and PCR reaction conditions. Because the cleavage occurs only if the probe hybridizes to the target, the origin of the detected fluorescence is specific amplification. The process of hybridization and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridizes to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis if SYBR green is used. By multiplexing, the target(s) and endogenous control can be amplified in single tube.

With Scorpion probes, sequence-specific priming and PCR product detection is achieved using a single oligonucleotide. The Scorpion probe maintains a stem-loop configuration in the unhybridized state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA. Disadvantages of SYBR green-based real-time PCR include the requirement for extensive optimization. Furthermore, non-specific amplifications require follow-up assays (melting point curve or dissociation analysis) for amplicon identification. The method has been used in HFE-C282Y genotyping. Another controllable problem is that longer amplicons create a stronger signal (if combined with other factors, this may cause CDC camera saturation, see below). Normally SYBR green is used in singleplex reactions, however when coupled with melting point analysis, it can be used for multiplex reactions.

The threshold cycle or the C_{T} value is the cycle at which a significant increase in ΔRn is first detected (for definition of ΔRn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10^{(-1/slope)}−1. The efficiency of the PCR should be 90-100% (3.6>slope>3.1). A number of variables can affect the efficiency of the PCR. These factors include length of the amplicon, secondary structure and primer quality. Although valid data can be obtained that fall outside of the efficiency range, the qRT-PCR should be further optimized or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterized by gradual increase or decrease in fluorescence without amplification of the product.) The important parameter for quantitation is the C_{T}. The higher the initial amount of genomic DNA, the sooner accumulated product is detected in the PCR process, and the lower the C_{T} value. The threshold should be placed above any baseline activity and within the exponential increase phase (which looks linear in the log transformation). Some software allows determination of the cycle threshold (C_{T}) by a mathematical analysis of the growth curve. This provides better run-to-run reproducibility. A C_{T} value of 40 means no amplification and this value cannot be included in the calculations. Besides being used for quantitation, the C_{T} value can be used for qualitative analysis as a pass/fail measure.

Multiplex TAQMAN® brand assays can be performed using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

III.C. Nested PCR

The disclosed methods can further utilize nested PCR. Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction. Nested PCR is often more successful in specifically amplifying long DNA fragments than conventional PCR, but it requires more detailed knowledge of the target sequences.

III.D. Primers and Probes

As used herein, “primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, “probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

Disclosed are compositions including primers and probes, which are capable of interacting with the disclosed COMT, OPRM1, and/or DAT1 nucleic acids or its complement. In certain embodiments the primers are used to support nucleic acid extension reactions, nucleic acid replication reactions, and/or nucleic acid amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are disclosed. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids. As an example of the use of primers, one or more primers can be used to create extension products from and templated by a first nucleic acid.

The size of the primers or probes for interaction with the nucleic acids can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the nucleic acid of interest typically will be used to produce extension products and/or other replicated or amplified products that contain a region of the nucleic acid of interest. The size of the product can be such that the size can be accurately determined to within 3, or 2 or 1 nucleotides.

In certain embodiments the product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product can be, for example, less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Thus, it is understood and herein provided that the disclosed RT-PCR and PCR reactions that comprise a portion of the disclosed methods or performed using the disclosed kits require forward and reverse primers to form a primer pair. Herein disclosed, the forward and reverse primer pair in the disclosed methods and kits can be SEQ ID NO: 1 or SEQ ID NO: 3; and SEQ ID NO: 2.

It is understood and herein contemplated that there are situations where it may be advantageous to utilize more than one primer pair to detect the presence of a fusion, truncation, or over expression mutation. Such RT-PCR or PCR reactions can be conducted separately, or in a single reaction. When multiple primer pairs are placed into a single reaction, this is referred to as “multiplex PCR.” For example, the reaction can comprise a first COMT and/or OPRM1 forward and reverse primer pair, as well as, second COMT and/or OPRM1 forward and reverse primer. In some instances, the second forward and reverse primer can be internal (i.e., nested) to the first COMT and/or OPRM1 forward and reverse primer.

Thus, disclosed herein in some embodiments are methods of detecting a susceptibility to opioid antagonist therapy or treating a psychiatric, mental, and/or neurological disorder (such as, for example, alcohol use disorder (AUD)), assaying the nucleic acid from a subject for the genotype of the dopamine (DA)-catabolizing enzyme catechol-O-methyltransferase (COMT) and the genotype of the opioid mu receptor gene (OPRM1) further comprising assaying DNA and RNA in a tissue sample for the number genotype of alleles coding for the number of VNTR repeats comprising wherein the PCR reaction a primer pair capable of specifically hybridizing to one or more DAT1 sequences for example, 5′-TGTGGTGTAGGGAACGGCCTGAG-3′ (SEQ ID NO: 1) and 5′-CTTCCTGGAGGTCACGGCTCAAGG-3′ (SEQ ID NO: 2). An alternative forward primer is 5′-TGCGGTGTAGGGAACGGCCTGAG-3′ (SEQ ID NO: 3).

III.E. Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TAQMAN® brand probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TAQMAN® brand probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are similar to cleavage activated probes except that they are primers that are incorporated into replicated strands and are then subsequently cleaved.

III.F. Labels

To aid in detection and quantitation of nucleic acids produced using the disclosed methods, labels can be directly incorporated into nucleotides and nucleic acids or can be coupled to detection molecules such as probes and primers. As used herein, a label is any molecule that can be associated with a nucleotide or nucleic acid, directly or indirectly, and which results in a measurable, detectable signal, either directly or indirectly. Many such labels for incorporation into nucleotides and nucleic acids or coupling to nucleic acid probes are known to those of skill in the art. Examples of labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, CASCADE BLUE®, OREGON GREEN®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

The absorption and emission maxima, respectively, for some of these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J., United States of America; Molecular Probes, Eugene, Oreg.; and Research Organics, Cleveland, Ohio., United States of America.

Additional labels of interest include those that provide for signal only when the probe with which they are associated is specifically bound to a target molecule, where such labels include: “molecular beacons” as described in Tyagi & Kramer, 1996 and European Patent Publication EP 0 070 685 B1. Other labels of interest include those described in U.S. Pat. No. 5,563,037 which is incorporated herein by reference.

Labeled nucleotides are a form of label that can be directly incorporated into the amplification products during synthesis. Examples of labels that can be incorporated into amplified nucleic acids include nucleotide analogs such as BrdUrd, aminoallyldeoxyuridine, 5-methylcytosine, bromouridine, and nucleotides modified with biotin or with suitable haptens such as digoxygenin. Suitable fluorescence-labeled nucleotides are Fluorescein-isothiocyanate-dUTP, Cyanine-3-dUTP and Cyanine-5-dUTP. One example of a nucleotide analog label for DNA is BrdUrd (bromodeoxyuridine, BrdUrd, BrdU, BUdR, Sigma-Aldrich Co). Other examples of nucleotide analogs for incorporation of label into DNA are AA-dUTP (aminoallyl-deoxyuridine triphosphate, Sigma-Aldrich Co.), and 5-methyl-dCTP (Roche Molecular Biochemicals). One example of a nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these labels are also considered labels. Any of the known labels can be used with the disclosed probes, tags, and method to label and detect nucleic acid amplified using the disclosed method. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody. As used herein, detection molecules are molecules which interact with amplified nucleic acid and to which one or more labels are coupled.

The disclosed methods can use any sequencing technique known in the art including, but not limited to methods disclosed by Sanger (dideoxy method), Maxam-Gilbert (chemical cleavage), automated sequencing (for example ABI systems) and next generation sequencing. From a technical perspective High-throughput or Next Generation Sequencing (NGS) represents an attractive option for detecting the somatic mutations within a gene. Unlike PCR, microarrays, high-resolution melting and mass spectrometry, which all indirectly infer sequence content, NGS directly ascertains the identity of each base and the order in which they fall within a gene. The newest platforms on the market have the capacity to cover an exonic region 10,000 times over, meaning the content of each base position in the sequence is measured thousands of different times. This high level of coverage ensures that the consensus sequence is extremely accurate and enables the detection of rare variants within a heterogeneous sample. For example, in a sample extracted from FFPE tissue, relevant mutations are only present at a frequency of 1% with the wild-type allele comprising the remainder. When this sample is sequenced at 10,000× coverage, then even the rare allele, comprising only 1% of the sample, is uniquely measured 100 times over. Thus, NGS can provide reliably accurate results with very high sensitivity, making it ideal for clinical diagnostic testing of FFPEs and other mixed samples.

Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing. MPSS was a bead-based method that used a complex approach of adapter ligation followed by adapter decoding, reading the sequence in increments of four nucleotides; this method made it susceptible to sequence-specific bias or loss of specific sequences. Polony sequencing, combined an in vitro paired-tag library with emulsion PCR, an automated microscope, and ligation-based sequencing chemistry to sequence an E. coli genome at an accuracy of >99.9999% and a cost approximately 1/10 that of Sanger sequencing.

A parallelized version of pyrosequencing, the method amplifies DNA inside water droplets in an oil solution (emulsion PCR), with each droplet containing a single DNA template attached to a single primer-coated bead that then forms a clonal colony. The sequencing machine contains many picolitre-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

A sequencing technology based on reversible dye-terminators. DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion semiconductor sequencing is based on using standard sequencing chemistry, but with a novel, semiconductor based detection system. This method of sequencing is based on the detection of hydrogen ions that are released during the polymerization of DNA, as opposed to the optical methods used in other sequencing systems. A micro well containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run.

Helicos's single-molecule sequencing uses DNA fragments with added polyA tail adapters, which are attached to the flow cell surface. The next steps involve extension-based sequencing with cyclic washes of the flow cell with fluorescently labeled nucleotides (one nucleotide type at a time, as with the Sanger method). The reads are performed by the Helioscope sequencer.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution.

The wells are constructed in a way that only the fluorescence occurring by the bottom of the well is detected. The fluorescent label is detached from the nucleotide at its incorporation into the DNA strand, leaving an unmodified DNA strand.

Single molecule real time sequencing based on RNA polymerase (RNAP), which is attached to a polystyrene bead, with distal end of sequenced DNA is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types (similarly to Sangers method).

Nanopore sequencing is based on the readout of electrical signal occurring at nucleotides passing by alpha-hemolysin pores covalently bound with cyclodextrin. The DNA passing through the nanopore changes its ion current. This change is dependent on the shape, size and length of the DNA sequence. Each type of the nucleotide blocks the ion flow through the pore for a different period of time.

VisiGen Biotechnologies uses a specially engineered DNA polymerase. This polymerase acts as a sensor—having incorporated a donor fluorescent dye by its active center. This donor dye acts by FRET (fluorescent resonant energy transfer), inducing fluorescence of differently labeled nucleotides. This approach allows reads performed at the speed at which polymerase incorporates nucleotides into the sequence (several hundred per second). The nucleotide fluorochrome is released after the incorporation into the DNA strand.

Sequencing by hybridization is a non-enzymatic method that uses a DNA microarray. A single pool of DNA whose sequence is to be determined is fluorescently labeled and hybridized to an array containing known sequences. Strong hybridization signals from a given spot on the array identify its sequence in the DNA being sequenced.

Mass spectrometry may be used to determine mass differences between DNA fragments produced in chain-termination reactions.

Another NGS approach is sequencing by synthesis (SBS) technology which is capable of overcoming the limitations of existing pyrosequencing based NGS platforms.

Such technologies rely on complex enzymatic cascades for read out, are unreliable for the accurate determination of the number of nucleotides in homopolymeric regions and require excessive amounts of time to run individual nucleotides across growing DNA strands. The SBS NGS platform uses a direct sequencing approach to produce a sequencing strategy with very a high precision, rapid pace and low cost.

SBS sequencing is initialized by fragmenting of the template DNA into fragments, amplification, annealing of DNA sequencing primers, and finally affixing as a high-density array of spots onto a glass chip. The array of DNA fragments are sequenced by extending each fragment with modified nucleotides containing cleavable chemical moieties linked to fluorescent dyes capable of discriminating all four possible nucleotides. The array is scanned continuously by a high-resolution electronic camera (Measure) to determine the fluorescent intensity of each base (A, C, G or T) that was newly incorporated into the extended DNA fragment. After the incorporation of each modified base the array is exposed to cleavage chemistry to break off the fluorescent dye and end cap allowing additional bases to be added. The process is then repeated until the fragment is completely sequenced or maximal read length has been achieved.

### IV. Nucleic Acids

There are a variety of molecules disclosed herein that are nucleic acid based, including for example the nucleic acids that encode, for example primers that hybridize to COMT and/or OPRM1. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

III.A. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., 1989). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

IV.B. Primers and Probes

Disclosed are compositions including primers and probes, which are capable of interacting with the disclosed nucleic acids, such as the COMT primers, OPRM1 primers, and/or DAT1 primers (SEQ ID NO: 1 or SEQ ID NO: 3; and SEQ ID NO: 2) as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the COMT, OPRM1 and/or DAT1 gene typically will be used to produce an amplified DNA product that contains a region of the COMT, OPRM1 and/or DAT1 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### V. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for treating a psychiatric, mental, and/or neurological disorder (such as, for example, alcohol use disorder (AUD)) or detecting susceptibility to opioid receptor antagonist treatment disclosed herein, comprising the primers that specifically hybridize to COMT (more preferably are able to specifically hybridize to COMT and able to amplify the nucleic acids encoding amino acid 158) and OPRM1 (more preferably are able to specifically hybridize to OPRM1 and able to amplify the nucleic acids encoding amino acid 40). In some embodiments, the disclosed kits for treating a psychiatric, mental, and/or neurological disorder (such as, for example, alcohol use disorder (AUD)) can also comprise an opioid receptor antagonist (such as, for example naltrexone).

### EXAMPLES

The following EXAMPLES are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Introduction to the Examples

### Opioid and Dopamine Genes Interact to Predict Naltrexone Response in an Alcohol Use Disorder Clinical Trial

While the opiate antagonist, naltrexone, is approved for Alcohol Use Disorder (AUD), not everyone benefits. These Examples evaluated whether the OPRM1 SNP rs1799971 interacts with the dopamine transport gene DAT1/SLC6A3 VNTR rs28363170 or the catechol-o-methyltransferase (COMT) gene SNP rs4680 in predicting naltrexone response.

DSM-IV alcohol dependent individuals were randomly assigned to naltrexone (50 mg/day) or placebo based on their OPRM1 A118G genotype (75 G allele carriers and 77 A allele homozygotes) who were also genotyped for DAT1 VNTR (9 vs 10 repeats) or COMT 158 SNP (Val/Val (A,A alleles) vs. Met (at least one G allele) carriers). Heavy drinking days (% HDD) were evaluated over 16 weeks and at the end of treatment. Effect sizes (d) for naltrexone response were calculated based on genotypes.

Naltrexone, relative to placebo, significantly reduced % HDD among OPRM1 G carriers who also had DAT1 10/10 (p=0.021; d=0.72) or COMT Val/Val genotypes (p=0.05; d=0.80), and to a similar degree in those OPRM1 A homozygotes who were also DAT1 9-repeat carriers (p=0.09; d=0.70) or COMT met carriers (p=0.03; d=0.63). Other genotype combinations did not respond differentially to naltrexone treatment. Smoking status did not materially affect the pharmacogenetic findings.

While not wishing to be bound by any particular theory of operation, AUD individuals with more opioid responsive genotypes (OPRM1 G carriers) might respond better to naltrexone because they have normal/less dopamine tone promoting genotypes (DAT1 10/10 or COMT Val/Val) while those with less responsive opioid responsive genotypes (OPRM1 A homozygotes) might respond better to naltrexone because they have dopamine enhancing phenotypes (DAT1 9-repeat or COMT Met carriers). These results could lead to more personized treatment approaches.

The following Examples provide results from an expanded analysis of a single site prospective study in which individuals with DSM-IV alcohol dependence were prospectively genotyped for their OPRM1 Al 18G status (G-allele carriers vs. A-allele homozygotes) and then randomized to receive naltrexone or placebo (Schacht et al., 2017). In a pre-planned analysis, these same individuals were also genotyped for the DAT1/SLC6A3 VNTR and COMT Val158Met SNP to evaluate the epistatic effects of these genotypes with OPRM1 A118G genotype on naltrexone efficacy.

### Materials and Methods for the Examples

Trial Design and Participants. The study was a 16-week randomized clinical trial (clinicaltrials.gov #NCT00920829) of naltrexone vs. placebo with initial stratification by OPRM1 A118G genotype (A-allele homozygotes versus G-allele carriers). Participants were between 18-70 years of age, met DSM-IV criteria for alcohol dependence by SCID interview (First et al., Structured Clinical Interview for DSM-IV Axis I Disorders—Patient Edition. SCID-I/P, Version 2.0 ed. New York, NY: New York State Psychiatric Institute; 1997), could not be of African American descent by self-report (due to low frequency of G alleles), consumed, on average, at least 5 drinks per day prior to screening, and were required to be abstinent at least 4 days prior to randomization. Participants did not meet DSM-IV criteria for dependence for any other drug but nicotine. Cocaine or marijuana use prior to screening was allowable, but no psychoactive drug could be evident in the urine at screening. Participants were not taking psychotropic medications other than antidepressants (stable dose for one month required) and did not meet criteria for current major depression, bipolar disorder, psychoses, PTSD, or eating disorders. They had to be medically stable (not having liver enzymes, ALT and AST, more than 3 times normal). Females were required to use a reliable form of contraception or be post-menopausal, and were not pregnant or nursing.

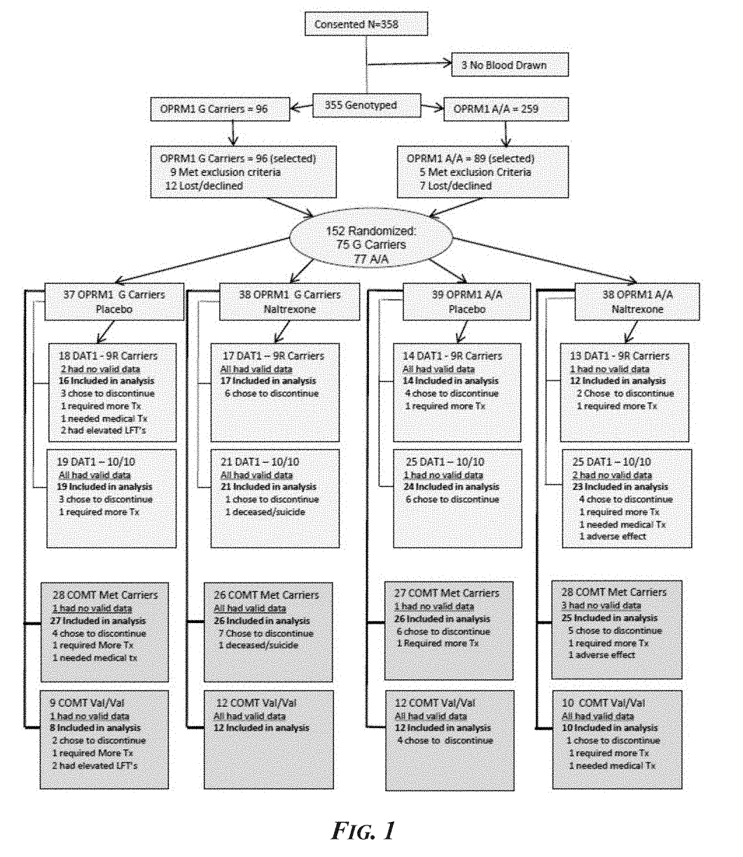

Recruitment. The study was approved by the Institutional Review Board (IRB) at the Medical University of South Carolina (Charleston, S.C., United States of America) and participants signed informed consent before formal assessment and genotyping. Participants, recruited primarily by community advertisement, weren't currently receiving other alcohol treatment. After genotyping (blind to subjects and study staff), individuals with at least one G allele and approximately ⅓ of A-allele homozygotes (temporally proximal to G allele carriers) were offered participation. Subsequently, pre-planned genotyping for the DAT1 VNTR and COMT Val158Met SNP was conducted to form the combined gene groups for this evaluation (see FIG. 1, CONSORT Diagram).

Medication. After at least 4 days of abstinence, participants were randomized to receive naltrexone (25 mg for 2 days, then 50 mg thereafter) or identical placebo (distributed in labeled blister packs) for 16 weeks.

Assessment and Medical Management (MM). Medical management (MM), a manualized intervention (Pettinati et al., 2005) designed and used previously (Anton et al., 2006b), and also utilized in previous naltrexone pharmacogenetic studies (Oslin et al., 2003; Anton et al., 2008) was administered. During each MM session (conducted on weeks 1, 2, 3, 4, 6, 8, 10, 12, 16) medication adverse effects using the SAFTEE (Johnson et al., 2005) were collected, compliance reviewed and motivated. Participants were encouraged, but not required, to attend Alcoholics Anonymous meetings.

Assessment. Multiple assessments were administered prior to randomization, including: SCID-IV, Alcohol Dependence Scale (ADS; Skinner & Allen, 1982), Obsessive Compulsive Drinking Scale (OCDS; Anton et al., 1996), Form-90 (modified time-line followback method for documenting alcohol consumption; Tonigan et al., 1997), Drinker Inventory of Consequences (DrInC), the Clinical Institute Withdrawal Assessment for Alcohol-Revised (Forcehimes et al., 2007), and baseline physical complaints. Lab tests included a health screen, liver function tests, pregnancy test (females) and alcohol use markers gamma-glutamyltransferase (GGT) and carbohydrate-deficient transferrin (Litten et al., 2010; Helander et al., 2010; Anton et al., 2006a).

During each study visit, the calendar based time line follow-back (Sobell & Sobell, 2000) was used to assess daily drinking. Study drop-outs were paid ($50) to return at week 16 for drinking assessment.

Outcome Measures. The primary a priori defined outcome measure was percent heavy drinking days (5 or more standard drinks per day for men and 4 or more for women) per month, over the course of treatment.

Genetic and Biological Tests/Assays. The Clinical Neurobiology Lab (CNL) at Medical University of South Carolina (MUSC; Raymond Anton, Director) extracted genomic DNA from peripheral blood mononuclear cells using a commercial DNA extraction kit/procedure (Qiagen Inc., Valencia, Calif., United States of America). OPRM1 A118G (Asn40 vs. Asp40 alleles) and COMT 158 (Val vs. Met alleles) were determined by a 5′ nuclease genotyping assay (commercially available under the registered trademark TAQMAN®) using primers and allele-specific probes (Catalogue No. C_8950074-1; Applied Biosystems, Foster City, Calif., United States of America. Samples previously genotyped in the laboratory of Dr. David Goldman (NIAAA Intramural) and in the CNL were used as controls (3 each per genotype/assay). The DAT1 VNTR was assessed after PCR amplification by specific primers 5′-TGTGGTGTAGGGAACGGCCTGAG-3′ (SEQ ID NO: 1) and 5′-CTTCCTGGAGGTCACGGCTCAAGG-3′ (SEQ ID NO: 2) (Thermo Fisher Scientific, Waltham, Mass., United States of America). Amplified samples were electrophoresed on 2.0% agarose gels and visualized with ethidium bromide under ultraviolet light. Previously identified and sequenced controls for each VNTR length (9 or 10 repeats) were amplified in each assay and two raters compared them with subject samples to call genotypes. Four subjects who carried an 11-repeat allele were reclassified as 10 carriers for this analysis. For each polymorphism, genotypes were in Hardy-Weinberg equilibrium and consistent with published frequencies among individuals of European American descent (Kang et al., 1999; Palmatier et al., 1999). RA supervised quality control in a blinded fashion. %CDT was measured with a reference HPLC assay (Helander et al., 2010), other blood chemistries including GGT were measured with an auto-analyzer.

Sample Size Estimates. Sample size estimates for the original study were guided by power analysis based on the magnitude of the OPRM1 by medication interaction on heavy drinking days in the COMBINE Study (Anton et al., 2008). The sample size for this exploratory epistatic evaluation was limited by that collected for the original OPRM1 gene evaluation (Schacht et al., 2017).

Randomization. Participating subjects were entered into a pre-constructed randomization program based on OPRM1 genotype (strata) with the following URN randomization variables: sex, smoking status, alcohol use disorder family history, antidepressant, and recent cocaine use. The program assigned them to a study medication group based on a priori determination. All subjects, assessment and treatment staff, were blind to both genotype and medication assignment. Since DAT1 and COMT genotyping were done later they did not serve as randomization variables.

Statistical Analysis. Chi-Square or ANOVA were used to evaluate differences in baseline demographic and alcohol use data between the 2 combinations of 8 treatment groups: medication (naltrexone vs. placebo)×OPRM1 genotype (A-allele homozygotes vs. G-allele carriers)×either DAT1 genotype [10R homozygotes vs. 9R carriers] or COMT genotype (Val allele homozygotes vs. Met allele carriers; see Table 1). Two baseline predictors of percent heavy drinking days during the trial, “current employment” and “time since last drink prior to randomization”, were used as covariates in the intent-to-treat (ITT) analysis (all subjects with at least 1 week of drinking data) using a linear mixed model (SPSS ver.22, —Linear Mixed, IBM, Armonk, N.Y., United States of America) with an unstructured variance/covariance matrix focusing on 4 bins of monthly percent heavy drinking data with medication group, time, OPRM1 genotype, and either DAT1 or COMT genotypes as factors. Based on previous data (Anton et al., 2008 and Schacht et al., 2017), the initial protocol stipulated that the last month of treatment would be analyzed independently as contrasts within the mixed model. Effect sizes of naltrexone compared to placebo were calculated for each gene combination for comparative/illustrative purposes. Since nicotine use was found to affect naltrexone response (Schacht et al., 2017; Anton et al., 2018), a sensitivity analysis was performed to evaluate the effect of smoking as a factor in the main ITT analysis.

### Example 1

### Randomization and Baseline Characteristics

Of those initially screened, 358 individuals consented to participate, and 152 were randomized with equal distribution between OPRM1 gene groups (see FIG. 1). Since 6 did not have valid outcome data, there were 146 evaluable individuals. Of those, 40 terminated the study early (similar across all gene/medication groups in numbers and reasons), but full 16-week drinking data was available on 126 (range 84-91% across gene/medication groups). Determined by pill count, 82% of subjects took at least 80% of study medication, again similar across all groups.

A 2 OPRM1 gene×2 allele (independently for DAT1 and COMT alleles)×2 medication group ANOVA showed there were no statistical differences between genotype/medication groups across a range of demographic and drinking variables, including sex, age, nicotine use, antidepressant and cocaine use, baseline drinking, biomarker, and severity measures (Table 1).

### Example 2

### Drinking Outcome Measures

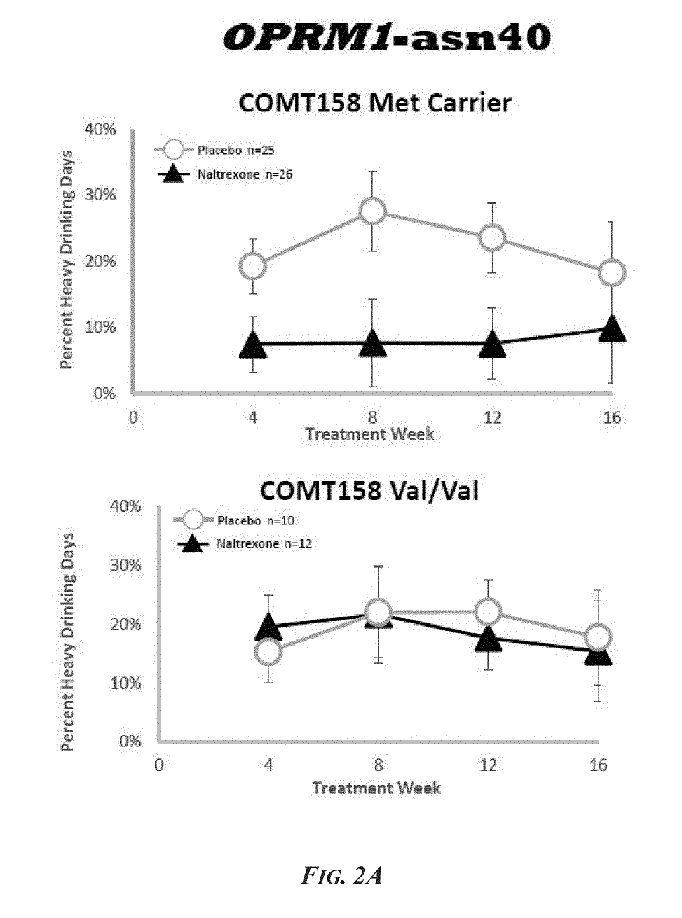

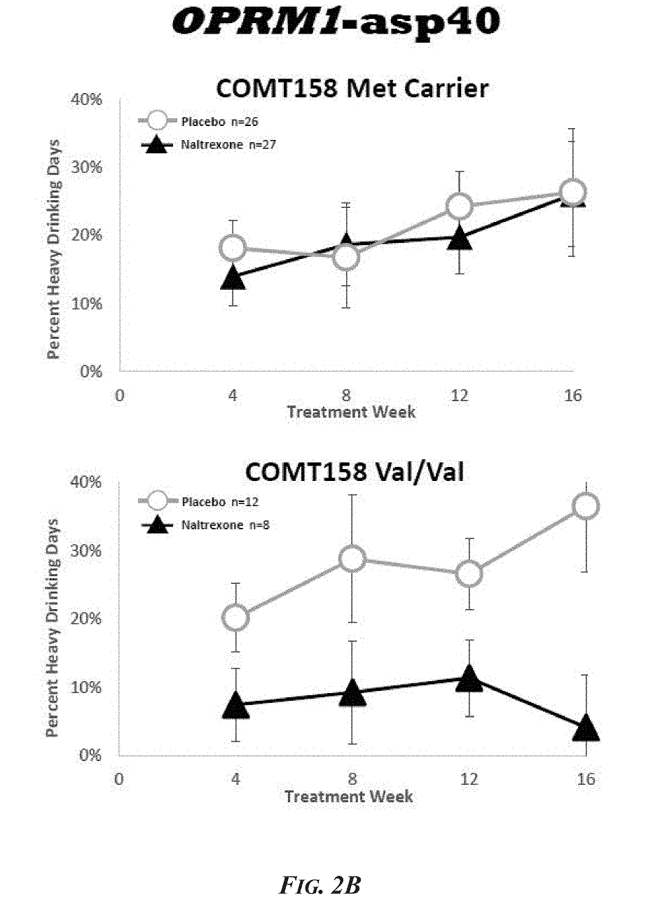

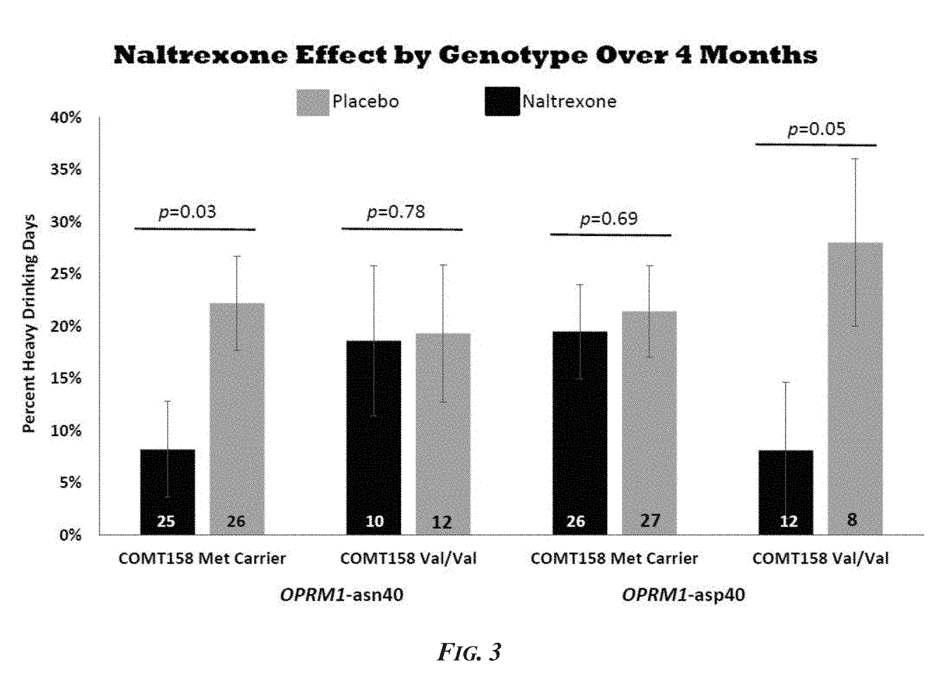

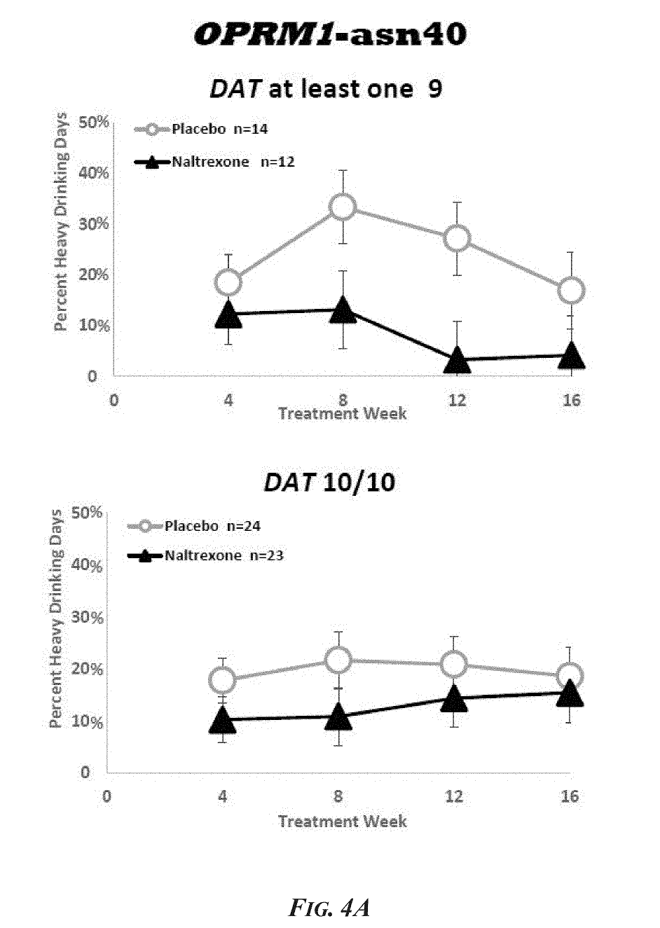

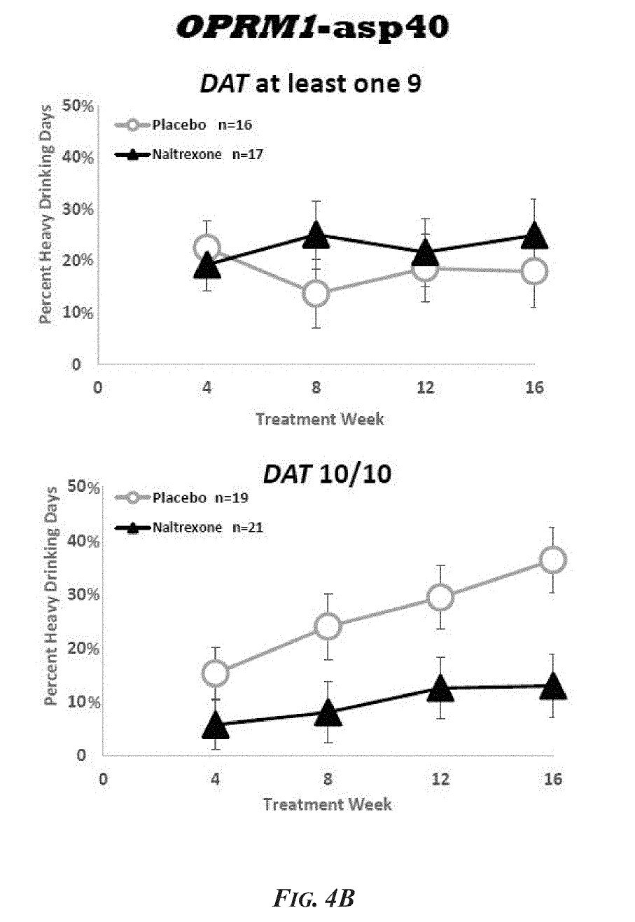

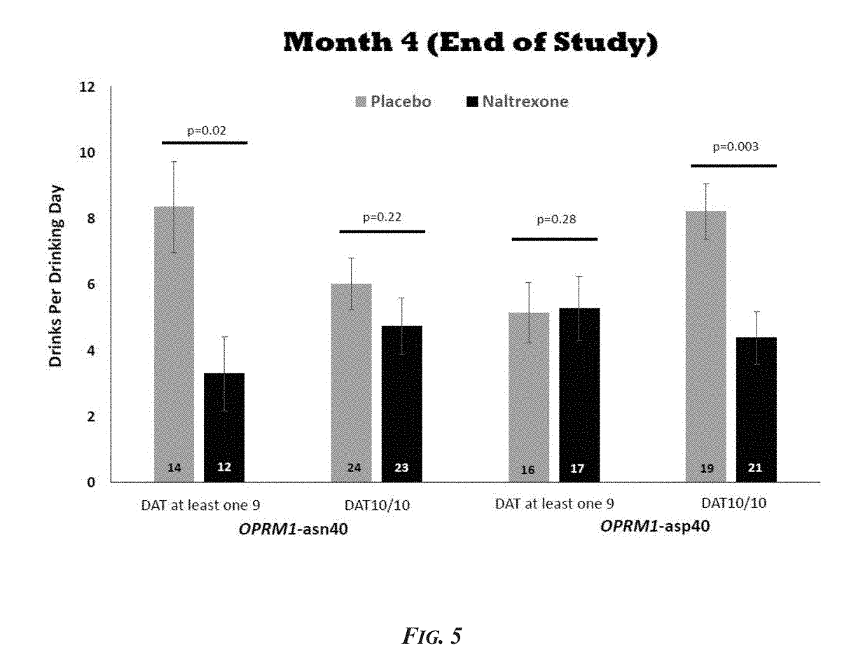

The main ITT analysis on percent heavy drinking days (HDD %) included the evaluable participants in each group for the combination of OPRM1×COMT (see FIGS. 2 and 3) or OPRM1×DAT1 (see FIGS. 4 and 5).

As shown in FIG. 2A, COMT158 Met carriers (i.e., those subjects whose genomes had one or two COMT rs4680 alleles that encoded methionine at amino acid 158 of SEQ ID NO: 8; also referred to herein as the “COMT A allele”) responded better to naltrexone when their genomes also included two copies of the OPRM1 asparagine allele (i.e., the allele that has an adenine at nucleotide position 561 of SEQ ID NO: 11; also referred to herein as the “OPRM1 A allele” or the “OPRM1-Asn40 allele”). However, if the subject had at least one copy of the OPRM1 aspartic acid allele (i.e., the allele that has a guanine at nucleotide position 561 of SEQ ID NO: 11; also referred to herein as the “OPRM1 G allele” or the “OPRM1-Asp40 allele”), those subjects who were homozygous for the COMT158 Val allele (i.e., the allele that has a guanine at nucleotide position 721 of SEQ ID NO: 7; also referred to herein as the “COMT G allele”) responded better to naltrexone (see FIG. 2B). FIG. 3 also shows that the response of COMT158 Met carriers whose genomes also included two copies of the OPRM1 asparagine allele was statistically significantly different from placebo (p=0.03) and the response of COMT158 Val/Val homozygotes whose genomes also included at least one copy of the OPRM1 aspartic acid allele was statistically significantly different from placebo (p=0.05)

The interactions among OPRM1 and DAT1 VNTR genotypes was also examined. As shown in FIG. 4A, those subjects who were homozygous for the OPRM1 asparagine allele responded better to naltrexone when their genomes also included two copies of the DAT1 VNTR 9R allele (i.e., those subjects whose genomes had one or two copies of the DAT1 VNTR 9-repeat allele). However, if the subject had at least one copy of the OPRM1 aspartic acid allele, those subjects who were homozygous for the DAT1 VNTR 10-repeat allele (i.e., those subjects whose genomes had two copies of the DAT1 VNTR 10-repeat allele) responded better to naltrexone (see FIG. 4B). FIG. 5 also shows that the response of DAT1 VNTR 9R carriers whose genomes also included two copies of the OPRM1 asparagine allele was statistically significantly different from placebo (p=0.02) and the response of DAT1 VNTR 1OR homozygotes whose genomes also included at least one copy of the OPRM1 aspartic acid allele was statistically significantly different from placebo (p=0.003)

In order to compare genotype by medication differences across the spectrum of genotypes, and to illustrate the clinical significance of the pharmacogenetic interactions, the univariate analyses in each genotype group and the effect size of naltrexone over placebo, for the full 16 weeks of treatment as well as during the last 4 weeks of treatment, is given in Table 2.

Planned simple effects analyses showed that at the 16-week (end of study) time point, differences between the groups were generally in the same direction as the full study interval (see Table 2). However, for the OPRM1×DAT1 interaction, only those OPRM1 G-allele carriers, who also had the DAT1 10/10 genotype, were significantly more responsive to naltrexone than placebo (F=8.17, 1, 119, p=0.005). For the OPRM1×COMT interaction, only those OPRM1 G-allele carriers, who also had COMT Val/Val genotypes, were significantly more responsive to naltrexone than placebo (F=7.3, 1,123, p=0.008). As seen, while effect sizes remained large for the gene/medication groups discussed above, OPRM1 G-allele carriers who were also either DAT1 10R homozygotes or COMT Val/Val homozygotes benefitted the most from naltrexone, both during the course of the study and at the end of treatment.

Given our previous findings that nicotine use/smoking status might influence naltrexone response (Schacht et al., 2017; Anton et al., 2018) and despite similar nicotine use between all gene and medication groups (see Table 1), a sensitivity analysis, whereby nicotine-use was entered as a covariate into the ITT analyses, found that the pharmacogenetic results were not materially changed.

TABLE 1
Demographic, Alcohol Use, and Severity Measures

| | | | OPRM1x | OPRM1x |
|---|---|---|---|---|
| | Total | | DAT1 | COMT |
| | Sample | | Statistic^{a} | Statistic^{a} |
| Characteristic | (n = 146) | | P value | P value |
| Demographics | | | | |
| Age, y (mean (SD)) | 49.3 | (10.1) | 0.88 | 0.10 |
| Sex, M, No. (%) | 101 | (69.2) | 0.88 | 0.97 |
| Married, No. (%) | 94 | (64.4) | 0.33 | 0.12 |
| Employed, No. (%) | 114 | (78.1) | 0.16 | 0.74 |
| Education ≤ 12 years, No. (%) | 24 | (16.4) | 0.55 | 0.72 |
| Current Nicotine Use, No. (%) | 57 | (39.0) | 0.78 | 0.77 |
| Cocaine Use, No. (%) | 19 | (13.0) | 0.75 | 0.49 |
| Antidepressant use, No. (%) | 48 | (32.9) | 0.72 | 0.83 |
| Alcohol use and severity | | | | |
| indicators, mean (SD) | | | | |
| Drinking days (%)^{b} | 85.3 | (19.3) | 0.88 | 0.22 |
| Heavy drinking days (%)^{b} | 79.7 | (22.3) | 0.34 | 0.28 |
| Drinks per drinking day^{b} | 11.2 | (4.8) | 0.84 | 0.44 |
| Drinks per day^{b} | 9.6 | (5.0) | 0.88 | 0.39 |
| Days from last drink to | 6.9 | (4.4) | 0.13 | 0.44 |
| randomization | | | | |
| ADS score^{c} | 15.4 | (6.4) | 0.96 | 0.25 |
| OCDS score^{d} | 25.6 | (8.1) | 0.89 | 0.58 |
| Drinking consequences^{e} No. (%) | 41.4 | (18.6) | 0.54 | 0.23 |
| GGT > 63 IU/L | 46 | (31.5) | 0.62 | 0.32 |
| dCDT ≤ 1.7% No. (%)^{f} | 80 | (56) | 0.37 | 0.59 |

^{a}Test of differences across medication groups and two genes. ANOVA testing linear variables and Chi Square testing categorical variables.
^{b}Drinking calculated during the 30 days prior to screening.
^{c}Alcohol Dependence Scale
^{d}Obsessive Compulsive Drinking Scale
^{e}Drinker Inventory of Consequences
^{f}Disialo-Carbohydrate Deficient Transferrin - biomarker of heavy drinking

TABLE 2
Significance and Effect Sizes of the Interaction of Carious OPRM1
and DAT or COMT Genes and Naltrexone Response on Percent
Heavy Drinking Days over the Course of the Trial (16 weeks) and
at the End of the Study (last 4 weeks)

| | | | Treatment Effects | | | |
|---|---|---|---|---|---|---|
| | | | All 4 months | | Last Month | |
| | | | | Effect | | Effect |
| Genotypes | | N | p value* | Sizes** | p value* | Size** |
| OPRM1^{a} | DAT^{b} | | | | | |
| A/A | 9 Carr.^{c} | 26 | 0.085 | 0.70 | 0.25 | 0.46 |
| A/A | 10/10 | 47 | 0.4 | 0.25 | 0.82 | 0.07 |
| G Carriers | 9 Carr. | 33 | 0.62 | −0.17 | 0.5 | −0.24 |
| G Carriers | 10/10 | 40 | 0.021 | 0.72 | 0.005 | 0.89 |
| OPRM1 | COMT^{d} | | | | | |
| A/A | Met Carr. | 51 | 0.03 | 0.63 | 0.27 | 0.32 |
| A/A | Val/Val | 22 | 0.78 | −0.12 | 0.94 | −0.03 |
| G Carriers | Met Carr. | 53 | 0.69 | 0.11 | 0.92 | 0.03 |
| G Carriers | Val/Val | 20 | 0.05 | 0.80 | 0.008 | 1.09 |

*Univariate analysis testing the interaction of medication group (naltrexone × placebo) in each genetic combination.
**Effect size if for naltrexone over placebo in reduction of percent heavy drinking days
^{a}or G at the 118 position of the OPRM1 gene coding for Asn40 or Asp40 respectively
^{b}Number of 9 or 10 forty base pair repeats of the DAT1 gene
^{c}Carr.: carriers
^{d}Val or Met allele at the 158 position on the COMT gene

### Discussion of the Examples

While great emphasis has been placed on the OPRM1 Asp40 predicting naltrexone response, results have been inconsistent. One reason is that OPRM1 Asp40 might be interacting with other genetic variants (epistasis). One important possibility is the functional VNTR variant in the dopamine transporter coding (DAT1) gene. DAT1 VNTR 9R carriers to have lower DAT function leading to more synaptic dopamine availability underlying more reward and cue sensitivity. As shown herein OPRM1 Asn40/DAT1 9R carriers had less drinking on naltrexone that was also observed in OPRM1 Asp40/DAT1 10/10 homozygotes (i.e., Asp40/DAT 10/10 and Asn40/DAT 9 variants did best on naltrexone). Overall, this data indicates that genetically based dopamine system variation/tone can influence how opiate systems might respond and interact with medication. DAT 9 carriers putatively have lower DAT transport function and likely more synaptic dopamine. Accordingly, Asp40 carriers only respond to opioid receptor blockade if they have normal synaptic dopamine (DAT 10/10 carriers). Conversely, Asn40 homozygotes only respond to opioid receptor blockade if they have a downstream excess of synaptic dopamine (DAT 9 carriers). These results indicate that an epistatic balance of the opioid and dopamine systems exists and should be taken into account in AUD in regard to pharmacogenetic treatment response and personalized medicine initiatives.

As shown herein, OPRM1 Asn40/Met158 carriers had less drinking on naltrexone that was also observed in OPRM1 Asp40/COMT Val/Val158 carriers

146 Caucasian alcohol dependent individuals (DSM IV) with a mean 80% heavy drinking days (HDD) were randomized to naltrexone (50 mg) or placebo, based on their OPRM1 allele status (Asp40 vs. Asn40 homozygotes—TAQMAN® PCR) on a one to one basis in a blinded fashion. In an exploratory fashion DAT1 VNTR (using primers from ABI, PCR, and chromatographic separation) status was explored in these participants. Smoking (40%), antidepressant use (33%), and sex (30% Female) were equally distributed across groups. Nine sessions of Medical Management (wks. 1, 2, 3, 4, 6, 8, 10, 12, 16) were provided and drinking assessed by TLFB during the 16-week treatment. Percent heavy drinking days (% HDD) over the 4 study months were analyzed in mixed model of medication (ntx vs. plac.)×OPRM1 allele (at least one Asp40 allele vs. Asn/Asn homozygotes)×DAT1 (at least one 9 VNTR vs. 10/10 homozygotes)×time (study month) (FIG. 2). Drinks per Drinking Day (DPDD) in the end (month 4) of the trial was also explored (FIG. 5).

The overall study demographics and drinking characteristics are given in Table 1 of 146 evaluable individuals, n=73 were OPRM1 Asp40 carriers and 73 Asn40 homozygotes. DAT1 9 carriers (n=59) and 10/10 homozygotes (n=87) were not significantly different across OPRM1 and medication groups (group size range 12-24). There were no significant differences in any baseline demographic and drinking variables across the 8 study groups.

Of 146 evaluable individuals n=73 were OPRM1 Asp40 carriers and 73 Asn40 homozygotes. DAT1 9 carriers and 10/10 homozygotes were not significantly different across OPRM1 and medication groups (group size range 12-24). Study completers (73%) and complete drinking data (86%) did not differ across groups. There was a significant four-way interaction (p=0.015) such that those Asp40 carriers who also had DAT1 10/10 genotypes and those Asn40 genotypes that who were DAT1 9 carriers all had less % HDD over the course of the study when on naltrexone compared to placebo (FIGS. 2 and 5). This was not modified by sex, age, antidepressant use or smoking status. The same pharmacogenetic relationships held when percent days abstinent (p=0.022), drinks per day (p=0.051) and drinks per drinking day (p=0.024) were similarly analyzed.

Alcohol dependent individuals (DSM IV) with a mean 80% heavy drinking days (HDD) were randomized to naltrexone (50 mg) or placebo, based on their OPRM1 allele status (Asp40 vs. Asn40 homozygotes—TAQMAN® brand PCR) on a one to one basis in a blinded fashion. In an exploratory fashion COMT (using primers from ABI, PCR, and chromatographic separation) status was explored in these participants. Smoking (40%), antidepressant use (33%), and sex (30% Female) were equally distributed across groups. Nine sessions of Medical Management (wks. 1, 2, 3, 4, 6, 8, 10, 12, 16) were provided and drinking assessed by TLFB during the 16-week treatment. Percent heavy drinking days (% HDD) over the 4 study months were analyzed in mixed model of medication (naltrexone vs. placebo)×OPRM1 allele (at least one Asp40 allele vs. Asn/Asn homozygotes)×COMT (at least one Met158 allele vs. Val/Val homozygotes)×time (study month).

There was a significant four-way interaction (p=0.015) such that those Asp40 carriers who also had Val/Val158 genotypes and those Asn40 genotypes that who were Met158 carriers all had less % HDD over the course of the study when on naltrexone compared to placebo (FIGS. 3, 4A and 4B, and 5). This was not modified by sex, age, antidepressant use or smoking status. The same pharmacogenetic relationships held when percent days abstinent, drinks per day, and drinks per drinking day, were similarly analyzed.

These Examples had high internal validity, being conducted at one site with the same staff providing MM throughout the study, and using a priori OPRM1 genotyping and selection. Data collection was performed blind to all genotypes and medication group assignment. Individuals of clinical interest (some taking antidepressants and some having cocaine use) were included, and equally distributed across study groups. The analyses of DAT1 and COMT genotypes were hypothesis driven, and pre-planned, with the genotyping done prior to any OPRM1 outcome analyses.

In our initial report (Schacht et al., 2017), while there was a significant main effect of naltrexone over placebo overall, OPRM1 A118G status was not predictive of response in the ITT analysis (but did have some predictive value in the most adherent individuals). In the current expanded analysis of the epistatic interaction of the OPRM1 118 genotypes with several dopamine genes, very strong and significant medication by genotype interactions emerged. As the results show, those OPRM1 G allele carriers who were also DAT1 10,10 or COMT Val/Val homozygotes responded remarkably well to naltrexone compared to placebo (effect sizes of 0.7 and 0.8). In addition, those OPRM1 A/A homozygotes who were either DAT1 9R-carriers or COMT met-carriers also responded better to naltrexone than placebo (effect sizes 0.70 and 0.63 respectively). These effect sizes are much greater than the small to moderate effect sizes of naltrexone efficacy in unselected AUD individuals across many trials (Srisurapanont & Jarusuraisin, 2005; Maisel et al., 2013), and may explain the inconsistent results reported in past naltrexone/OPRM1 A118G pharmacogenetic studies. In this study, individuals with OPRM1 G allele who were DAT1 9R-carriers or COMT met-carriers or who were OPRM1 AA homozygotes who were DAT1 10,10 or COMT Val/Val homozygotes were, on average, naltrexone non-responders.

This pattern of epistatic interaction suggests that AUD individuals who are OPRM1 G carriers (those with putative increased endogenous opioid tone) might need to also have low/normal dopamine-tone, since genotypes normally reflective of heightened brain dopamine (DAT1 9R and COMT met) were not associated with naltrexone response in these individuals, while those genotypes that reflect low-normal dopamine tone (DAT1 1OR and COMT Val/Val) generally were most responsive.

It is possible that this interaction between endogenous opioid and dopamine systems in AUD (as extrapolated from functional genotyping) might provide valuable insights into the brain pathophysiology of AUD and suggest a way to unravel the complex effects of alcohol on brain transmitter systems. The fact that response to a targeted (and pharmacologically specific) medication like naltrexone could be differentially impacted by these interactions is both novel and noteworthy. It also suggests that multiple genes might need to be examined to provide the precision that personalized treatment of AUD might require, not a surprise given the biological and pharmacological response heterogeneity of the disorder.

The impact of nicotine-use/smoking on the pharmacogenetic findings was analyzed. It was found that nicotine-use/smoking did not materially impact the results presented here. However, future larger studies might further explore more subtle interactions of nicotine-use and genotype in AUD treatments.

Given the original hypothesis and sample size available, individuals could not be genotyped and subsequently randomized based on two/three genes, so some systematic selection and treatment assignment bias cannot be ruled out. Also, sample size limitation did not allow valid analysis of 4-way interactions between all three genotypes and medication response. Despite these limitations, the findings are underpinned by a well-executed clinical trial. Further, given the moderate expense of genotyping, clinicians might consider providing this service to AUD patients who might want to enhance their expectations of response to naltrexone and other opioid antagonist medications.

### REFERENCES

All references listed in the instant disclosure, including but not limited to all patents, patent applications and publications thereof, scientific journal articles, and database entries (including but not limited to GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Aarts et al. (2010) Neuropsychopharmacology 35(9):1943-1951.
Anton & Youngblood (2006) Alcohol Clin Exp Res 30(11):1878-1883.
Anton et al. (1996) Arch Gen Psychiatry 53:225-231.
Anton et al. (2006a) Alcohol Clin Exp Res 30(11):1878-1883.
Anton et al. (2006b) JAMA 295(17):2003-2017.
Anton et al. (2008) Arch Gen Psychiatry 65(2):135-144.
Anton et al. (2012) Alcohol Clin Exp Res 36(11):2000-2007.
Anton et al. (2018) Alcohol Clin Exp Res 42(4):751-760.
Chamorro et al. (2012) Addict Biol 17(3):505-512.
Chen et al. (2004) Am J Hum Genet 75(5):807-821.
Ciliax et al. (1999) J Comp Neurol 409(1):38-56.
Craddock et al. (2006) Mol Psychiatry 11(5):446-458.
Dreher et al. (2009) Proc Natl Acad Sci USA 106(2):617-622.
European Patent Publication EP 0 070 685 B1.
First et al. (1997) DSM-IV Axis I Disorders—Patient Edition. SCID-I/P, Version 2.0 ed. New York State Psychiatric Institute, New York, N.Y., United States of America.
Forbes et al. (2009) Mol Psychiatry 14(1):60-70.
Forcehimes et al. (2007) Addict Behav 32(8):1699-1704.
Fuke et al. (2001) Pharmacogenomics J 1(2):152-156.
Goldman et al. (2005) J Stud Alcohol Suppl. 2005 (15):56-64; discussion 33.
Gonzales & Weiss (1998) J Neurosci 18(24):10663-10671.
Grant et al. (2013) Eur Neuropsychopharmacol 23(11):1587-1596.
Grant et al. (2016) JAMA Psychiatry 73(1):39-47.
Greenfield et al. (2014) J Stud Alcohol Drugs 75(2):319-327.
Hasin et al. (2007) Arch Gen Psychiatry 64(7):830-842.
Heinz et al. (2000) Neuropsychopharmacology 22(2):133-139.
Helander et al. (2010) Clin Chem Lab Med 48(11):1585-1592.
Job et al. (2007) Biol Psychiatry 62(6):627-634.
Johnson et al. (2005) J Stud Alcohol Suppl Suppl 15(15):157-167; discussion 140.
Jones et al. (2015) Alcohol Clin Exp Res 39(3):391-402.
Kang et al. (1999) Biol Psychiatry 46(2):151-160.
Lachman (2008) Neuropsychopharmacology 33(13):3027-3029.
Lachman et al. (1996) Pharmacogenetics 6(3):243-250.
Letsinger et al. (1989) Proc Natl Acad Sci U S A 86:6553-6556.
Litten et al. (2010) Alcohol Clin Exp Res 34(6):955-967.
Maisel et al. (2013) Addiction 108(2):275-293.
Mark et al. (2009) Drug Alcohol Depend 99(1-3):345-349.
McCane et al. (2018) Alcohol 67:15-22.
Niciu & Arias (2013) CNS Drugs 27(10):777-787.
Oslin et al. (2003) Neuropsychopharmacology 28:1546-1552.
Oslin et al. (2015) JAMA Psychiatry 72(5):430-437.
Palmatier et al. (1999) Biol Psychiatry 46(4):557-567.
Pettinati et al. (2005) J Stud Alcohol Suppl 66(15):170-178; discussion 68-69.
Ramchandani et al. (2011) Mol Psychiatry 16(8):809-817.
Rehm et al. (2014) Alcohol Clin Exp Res 38(4):1068-1077.
Schacht (2016) Pharmacogenomics J 16(5):430-438.
Schacht et al. (2013) Neuropsychopharmacology 38(3):414-422.
Schacht et al. (2017) Neuropsychopharmacology 42(13):2640-2653.
Skinner & Allen (1982) J Abnorm Psychol 91(3):199-209.
Sobell & Sobell (2000) Alcohol Timeline Followback (TLFB). Handbook of Psychiatric Measures. American Psychiatric Association Publishing. Washington, D.C., United States of America pages 477-479.
Srisurapanont & Jarusuraisin (2005) Int J Neuropsychopharmacol 8(2):267-280.
Stewart et al. (2009) Am J Hypertens 22(1):87-91.
Sullivan et al. (1989) Br J Addict 84(11):1353-1357.
Tonigan et al. (1997) J Stud Alcohol 58(4):358-364.
Tyagi & Kramer (1996) Nature Biotechnology 14:303-308.
Zindel & Kranzler (2014) J Stud Alcohol Drugs Suppl 75 Suppl 17:79-88.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for treating a subject with a disorder associated with opioid receptor activity, the method comprising:
(a) performing or having performed one or more genotyping assays on nucleic acids isolated from the subject with respect to an opioid mu receptor (OPRM1) gene product and also a dopamine (DA)-catabolizing enzyme catechol-O-methyltransferase (COMT) gene product and/or a variable number tandem repeat (VNTR) polymorphism in the dopamine transporter gene DAT1/SLC6A3; and
(b) administering an opioid receptor antagonist therapy to the subject, wherein:
(i) the subject has at least one allele encoding a methionine at an amino acid position corresponding to amino acid residue 158 of the COMT gene product of SEQ ID NO: 8 and has two alleles encoding an asparagine at an amino acid position corresponding to amino acid residue 40 the of OPRM1 gene product of SEQ ID NO: 12; or
(ii) the subject is homozygous for a valine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(iii) the subject has at least one allele encoding a VNTR 9-repeat and has two alleles encoding an asparagine at an amino acid position corresponding to amino acid residue 40 the of OPRM1 gene product of SEQ ID NO: 12; or
(iv) the subject is homozygous for a VNTR 10-repeat and has at least one allele encoding an aspartic acid at an amino acid position corresponding to amino acid residue 40 of the OPRM1 gene product of SEQ ID NO: 12.

2. The method of claim 1, wherein the disorder associated with opioid receptor activity is an alcohol use disorder (AUD).

3. The method of claim 1, wherein at least one of the one or more genotyping assays is performed prior to administering the opioid receptor antagonist therapy to the subject.

4. The method of claim 1, wherein at least one of the one or more genotyping assays is performed after administering the opioid receptor antagonist therapy to the subject, and further wherein the opioid receptor antagonist therapy is discontinued if:
(v) the subject has at least one allele encoding a methionine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(vi) the subject is homozygous for a valine at an amino acid position corresponding to amino acid residue 158 of the COMT gene product of SEQ ID NO: 8 and is homozygous for an asparagine at an amino acid position corresponding to amino acid residue 40 the of OPRM1 gene product of SEQ ID NO: 12; or
(vi) the subject has at least one allele encoding a VNTR 9-repeat and has at least one allele encoding an aspartic acid at an amino acid position corresponding to amino acid residue 40 the of OPRM1 gene product of SEQ ID NO: 12; or
(viii) the subject is homozygous for a VNTR 10-repeat and is homozygous for an asparagine at an amino acid position corresponding to amino acid residue 40 of the OPRM1 gene product of SEQ ID NO: 12.

5. The method of claim 1, wherein the opioid receptor antagonist is naltrexone.

6. The method of claim 1, wherein the genotyping assay comprises a nucleic acid amplification process followed by sequencing or gel electrophoresis of an amplification product produced thereby.

7. A method for detecting susceptibility of a subject to an opioid receptor antagonist for a disorder associated with opioid receptor activity in the subject, the method comprising:
(a) obtaining a biological sample from the subject; and
(b) performing or having performed one or more genotyping assays on the biological sample from the subject with respect to an opioid mu receptor (OPRM1) gene product and also a dopamine (DA)-catabolizing enzyme catechol-O-methyltransferase (COMT) gene product and/or a variable number tandem repeat (VNTR) polymorphism in the dopamine transporter gene DAT1/SLC6A3;
wherein:
(i) the subject is susceptible to an opioid receptor antagonist if:
(a) the subject has at least one allele encoding a methionine at an amino acid position corresponding to amino acid residue 158 of the COMT gene product of SEQ ID NO: 8 and has two alleles encoding an asparagine at an amino acid position corresponding to amino acid residue 40 the of OPRM1 gene product of SEQ ID NO: 12; or
(b) the subject is homozygous for a valine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(c) the subject has at least one allele encoding a VNTR 9-repeat and has two alleles encoding an asparagine at an amino acid position corresponding to amino acid residue 40 the of OPRM1 gene product of SEQ ID NO: 12; or
(d) the subject is homozygous for a VNTR 10-repeat and has at least one allele encoding an aspartic acid at an amino acid position corresponding to amino acid residue 40 of the OPRM1 gene product of SEQ ID NO: 12; and
(ii) the subject is not susceptible to an opioid receptor antagonist if:
(e) the subject has at least one allele encoding a methionine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(f) the subject is homozygous for a valine at an amino acid position corresponding to amino acid residue 158 of the COMT gene product of SEQ ID NO: 8 and is homozygous for asparagine at an amino acid position corresponding to amino acid residue 40 the of OPRM1 gene product of SEQ ID NO: 12; or
(g) the subject has at least one allele encoding a VNTR 9-repeat and has at least one allele encoding an aspartic acid at an amino acid position corresponding to amino acid residue 40 the of OPRM1 gene product of SEQ ID NO: 12; or
(h) the subject is homozygous for a VNTR 10-repeat and is homozygous for an asparagine at an amino acid position corresponding to amino acid residue 40 of the OPRM1 gene product of SEQ ID NO: 12.

8. The method of claim 7, wherein the opioid receptor antagonist is naltrexone.

9. The method of claim 7, wherein at least one of the one or more genotyping assays comprises a nucleic acid amplification process followed by sequencing or gel electrophoresis of an amplification product produced thereby.

10. A method for identifying and treating a human subject having susceptibility to an opioid receptor antagonist therapy for a disorder associated with opioid receptor activity in the subject, the method comprising:
(a) obtaining a nucleic acid sample from a human subject;
(b) performing or having performed on the nucleic acid sample one or more genotyping assays to determine genotypes of the subject with respect to an opioid mu receptor (OPRM1) gene product and either or both of a dopamine (DA)-catabolizing enzyme catechol-O-methyltransferase (COMT) gene product and a variable number tandem repeat (VNTR) polymorphism in the dopamine transporter gene DAT1/SLC6A3; and
(c) administering an opioid receptor antagonist to the subject if the genotypes determined indicate that the subject:
(i) has at least one allele encoding a methionine at an amino acid position corresponding to amino acid residue 158 of the COMT gene product of SEQ ID NO: 8 and has two alleles encoding an asparagine at an amino acid position corresponding to amino acid residue 40 the of OPRM1 gene product of SEQ ID NO: 12; or
(b) is homozygous for a valine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(c) has at least one allele encoding a VNTR 9-repeat and has two alleles encoding an asparagine at an amino acid position corresponding to amino acid residue 40 the of OPRM1 gene product of SEQ ID NO: 12; or
(d) is homozygous for a VNTR 10-repeat and has at least one allele encoding an aspartic acid at an amino acid position corresponding to amino acid residue 40 of the OPRM1 gene product of SEQ ID NO: 12.

11. The method of claim 10, wherein the genotypes are determined prior to administering the opioid receptor antagonist.

12. The method of claim 11, wherein the genotypes are determined after an opioid receptor antagonist therapy has commenced and the opioid receptor antagonist therapy is discontinued if:
(e) the subject has at least one allele encoding a methionine at an amino acid position corresponding to amino acid residue 158 of the COMT gene product of SEQ ID NO: 8 and has at least one allele encoding an aspartic acid at an amino acid position corresponding to amino acid residue 40 of the OPRM1 gene product of SEQ ID NO: 12; or
(f) the subject is homozygous for a valine at an amino acid position corresponding to amino acid residue 158 of the COMT gene product of SEQ ID NO: 8 and has two alleles encoding an asparagine at an amino acid position corresponding to amino acid residue 40 the of OPRM1 gene product of SEQ ID NO: 12; or
(g) the subject has at least one allele encoding a VNTR 9-repeat and has at least one allele encoding an aspartic acid at an amino acid position corresponding to amino acid residue 40 the of OPRM1 gene product of SEQ ID NO: 12; or
(h) the subject is homozygous for a VNTR 10-repeat and has two alleles encoding an asparagine at an amino acid position corresponding to amino acid residue 40 of the OPRM1 gene product of SEQ ID NO: 12.

13. The method of claim 11, wherein the opioid receptor antagonist is naltrexone.

14. The method of claim 11, wherein at least one of the one or more genotyping assays comprises a nucleic acid amplification process followed by sequencing or gel electrophoresis of an amplification product produced thereby.

15. A method for treating a subject with a disorder associated with opioid receptor activity, the method comprising:
(a) performing or having performed one or more genotyping assays on nucleic acids isolated from the subject to determine the subject's genotype with respect to a first gene and a second gene, wherein the first gene is an opioid mu receptor (OPRM1) gene and the second gene is selected from the group consisting of a dopamine (DA)-catabolizing enzyme catechol-O-methyltransferase (COMT) gene and a dopamine transporter DAT1/SLC6A3 gene; and
(b) administering an opioid receptor antagonist therapy to the subject when:
(i) the subject's genotype has two alleles encoding an asparagine at an amino acid position corresponding to amino acid residue 40 the of OPRM1 gene product of SEQ ID NO: 12 and has at least one allele encoding a methionine at an amino acid position corresponding to amino acid residue 158 of the COMT gene product of SEQ ID NO: 8 or at least one allele encoding a dopamine transporter DAT1/SLC6A3 9-repeat variable number tandem repeat (VNTR); or
(ii) the subject's genotype has at least one allele encoding an aspartic acid at an amino acid position corresponding to amino acid residue 40 the of OPRM1 gene product of SEQ ID NO: 12 and has two alleles encoding a valine at an amino acid position corresponding to amino acid residue 158 of the COMT gene product of SEQ ID NO: 8 or two alleles encoding a dopamine transporter DAT1/SLC6A3 10-repeat variable number tandem repeat (VNTR).

16. The method of claim 15, wherein the disorder associated with opioid receptor activity is an alcohol use disorder (AUD).

17. The method of claim 15, wherein at least one of the one or more genotyping assays is performed prior to administering the opioid receptor antagonist therapy to the subject.

18. The method of claim 15, wherein at least one of the one or more genotyping assays are performed after administering the opioid receptor antagonist therapy to the subject, and further wherein the opioid receptor antagonist therapy is discontinued if:
(iii) the subject has at least one allele encoding an aspartic acid at an amino acid position corresponding to amino acid residue 40 of the OPRM1 gene product of SEQ ID NO: 12 and at least one allele encoding a methionine at an amino acid position corresponding to amino acid residue 158 of the COMT gene product of SEQ ID NO: 8 or at least one allele for a DAT1/SLC6A3 9-repeat variable number tandem repeat (VNTR); or
(iv) the subject is homozygous for an asparagine at an amino acid position corresponding to amino acid residue 40 the of OPRM1 gene product of SEQ ID NO: 12 and is homozygous for a valine at an amino acid position corresponding to amino acid residue 158 of the COMT gene product of SEQ ID NO: 8 or is homozygous for a DAT1/SLC6A3 10-repeat variable number tandem repeat (VNTR).

19. The method of claim 15, wherein the opioid receptor antagonist therapy comprises administering an effective amount of naltrexone to the subject.

20. The method of claim 15, wherein at least one of the one or more genotyping assays comprises a nucleic acid amplification process followed by sequencing or gel electrophoresis of an amplification product produced thereby.


SEQUENCE LISTING (12 sequences)

SEQ ID NO: 1 - DNA, 23 bp, Artificial Sequence
  Artificially synthesized oligonucleotide primer
tgtggtgtagggaacggcctgag

SEQ ID NO: 2 - DNA, 24 bp, Artificial Sequence
  Artificially synthesized oligonucleotide primer
cttcctggaggtcacggctcaagg

SEQ ID NO: 3 - DNA, 23 bp, Artificial Sequence
  Artificially synthesized oligonucleotide primer
tgcggtgtagggaacggcctgag

SEQ ID NO: 4 - DNA, 40 bp, Homo sapiens
actggagcgtgtactaccccaggacgcatgcagggccccc

SEQ ID NO: 5 - DNA, 51 bp, Homo sapiens
  misc_feature (26)..(26): r = a or g
ggtcaacttgtcccacttagatggcracctgtccgacccatgcggtccgaa

SEQ ID NO: 6 - DNA, 51 bp, Homo sapiens
  misc_feature (26)..(26): r = a or g
ccagcggatggtggatttcgctggcrtgaaggacaaggtgtgcatgcctga

SEQ ID NO: 7 - DNA, 2304 bp, Homo sapiens
  CDS (250)..(1065)
cggcctgcgtccgccaccggaagcgccctcctaatccccgcagcgccaccgccattgccgccatcgtcgtggggcttctggggcagctagggctgcccgccgcgctgcctgcgccggaccggggcgggtccagtcccgggcgggccgtcgcgggagagaaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggcccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccagcattcacacctttctgaccaagcaggcgctggggacaggtggaccccgcagcagcaccagcccctctgggccccatgtggcacagagtggaagcatctccttccctactccccactgggccttgcttacagaagaggcaatggctcagaccagctcccgcatccctgtagttgcctccctggcccatgagtgaggatgcagtgctggtttctgcccacctacacctagagctgtccccatctcctccaaggggtcagactgctagccacctcagaggctccaagggcccagttcccaggcccaggacaggaatcaaccctgtgctagctgagttcacctgcaccgagaccagcccctagccaagattctactcctgggctcaaggcctggctagcccccagccagcccactcctatggatagacagaccagtgagcccaagtggacaagtttggggccacccagggaccagaaacagagcctctgcaggacacagcagatgggcacctgggaccacctccacccagggccctgccccagacgcgcagaggcccgacacaagggagaagccagccacttgtgccagacctgagtggcagaaagcaaaaagttcctttgctgctttaatttttaaattttcttacaaaaatttaggtgtttaccaatagtcttattttggcttatttttaa

SEQ ID NO: 8 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 9 - DNA, 3952 bp, Homo sapiens
  CDS (127)..(1989)
cgctgcggagcgggaggggaggcttcgcggaacgctctcggcgccaggactcgcgtgcaaagcccaggcccgggcggccagaccaagagggaagaagcacagaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccctgcagggcagaaaaacgtctaacttcatgctgtctgtgtgaggctccctccctccctgctccctgctcccggctctgaggctgccccaggggcactgtgttctcaggcggggatcacgatccttgtagacgcacctgctgagaatccccgtgctcacagtagcttcctagaccatttactttgcccatattaaaaagccaagtgtcctgcttggtttagctgtgcagaaggtgaaatggaggaaaccacaaattcatgcaaagtcctttcccgatgcgtggctcccagcagaggccgtaaattgagcgttcagttgacacattgcacacacagtctgttcagaggcattggaggatgggggtcctggtatgtctcaccaggaaattctgtttatgttcttgcagcagagagaaataaaactccttgaaaccagctcaggctactgccactcaggcagcctgtgggtccttgcggtgtagggaacggcctgagaggagcgtgtcctatccccggacgcatgcagggcccccacaggagcgtgtcctatccccggacgcatgcagggcccccacaggagcatgtcctatccctggacgcatgcagggcccccacaggagcgtgtactaccccagaacgcatgcagggcccccacaggagcgtgtactaccccaggacgcatgcagggcccccactggagcgtgtactaccccaggacgcatgcagggcccccacaggagcgtgtcctatccccggaccggacgcatgcagggcccccacaggagcgtgtactaccccaggacgcatgcagggcccccacaggagcgtgtactaccccaggatgcatgcagggcccccacaggagcgtgtactaccccaggacgcatgcagggcccccatgcaggcagcctgcagaccacactctgcctggccttgagccgtgacctccaggaagggaccccactggaattttatttctctcaggtgcgtgccacatcaataacaacagtttttatgtttgcgaatggctttttaaaatcatatttacctgtgaatcaaaacaaattcaagaatgcagtatccgcgagcctgcttgctgatattgcagtttttgtttacaagaataattagcaatactgagtgaaggatgttggccaaaagctgctttccatggcacactgccctctgccactgacaggaaagtggatgccatagtttgaattcatgcctcaagtcggtgggcctgcctacgtgctgcccgagggcaggggccgtgcagggccagtcatggctgtcccctgcaagtggacgtgggctccagggactggagtgtaatgctcggtgggagccgtcagcctgtgaactgccaggcagctgcagttagcacagaggatggcttccccattgccttctggggagggacacagaggacggcttccccatcgccttctggccgctgcagtcagcacagagagcggcttccccattgccttctggggagggacacagaggacagcttccccatcgccttctggctgctgcagtcagcacagagagcggcttccccatcgccttctggggaggggctccgtgtagcaacccaggtgttgtccgtgtctgttgaccaatctctattcagcatcgtgtgggtccctaagcacaataaaagacatccacaatggaaaaactgcaaaaaaaaaaaaaaaaaa

SEQ ID NO: 10 - protein, 620 aa, Homo sapiens
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 11 - DNA, 15279 bp, Homo sapiens
  CDS (444)..(1646)
gctaccaaagactaactctatctctctccccaacccttctctccatctccctcctttagatgtgtttgcacagaagagtgcccagtgaagagacctactccttggatcgctttgcgcaaaatccaccccttttccctcctccctcccttccagcctccgaatcccgcatggcccacgctcccctcctgcagcggtgcggggcaggtgatgagcctctgtgaactactaaggtgggagggggctatacgcagaggagaatgtcagatgctcagctcggtcccctccgcctgacgctcctctctgtctcagccaggactggtttctgtaagaaacagcaggagctgtggcagcggcgaaaggaagcggctgaggcgcttggaacccgaaaagtctcggtgctcctggctacctcgcacagcggtgcccgcccggccgtcagtaccatggacagcagcgctgcccccacgaacgccagcaattgcactgatgccttggcgtactcaagttgctccccagcacccagccccggttcctgggtcaacttgtcccacttagatggcaacctgtccgacccatgcggtccgaaccgcaccgacctgggcgggagagacagcctgtgccctccgaccggcagtccctccatgatcacggccatcacgatcatggccctctactccatcgtgtgcgtggtggggctcttcggaaacttcctggtcatgtatgtgattgtcagatacaccaagatgaagactgccaccaacatctacattttcaaccttgctctggcagatgccttagccaccagtaccctgcccttccagagtgtgaattacctaatgggaacatggccatttggaaccatcctttgcaagatagtgatctccatagattactataacatgttcaccagcatattcaccctctgcaccatgagtgttgatcgatacattgcagtctgccaccctgtcaaggccttagatttccgtactccccgaaatgccaaaattatcaatgtctgcaactggatcctctcttcagccattggtcttcctgtaatgttcatggctacaacaaaatacaggcaaggttccatagattgtacactaacattctctcatccaacctggtactgggaaaacctgctgaagatctgtgttttcatcttcgccttcattatgccagtgctcatcattaccgtgtgctatggactgatgatcttgcgcctcaagagtgtccgcatgctctctggctccaaagaaaaggacaggaatcttcgaaggatcaccaggatggtgctggtggtggtggctgtgttcatcgtctgctggactcccattcacatttacgtcatcattaaagccttggttacaatcccagaaactacgttccagactgtttcttggcacttctgcattgctctaggttacacaaacagctgcctcaacccagtcctttatgcatttctggatgaaaacttcaaacgatgcttcagagagttctgtatcccaacctcttccaacattgagcaacaaaactccactcgaattcgtcagaacactagagaccacccctccacggccaatacagtggatagaactaatcatcagctagaaaatctggaagcagaaactgctccgttgccctaacagggtctcatgccattccgaccttcaccaagcttagaagccaccatgtatgtggaagcaggttgcttcaagaatgtgtaggaggctctaattctctaggaaagtgcctgcttttaggtcatccaacctctttcctctctggccactctgctctgcacattagagggacagccaaaagtaagtggagcatttggaaggaaaggaatataccacaccgaggagtccagtttgtgcaagacacccagtggaaccaaaacccatcgtggtatgtgaattgaagtcatcataaaaggtgacccttctgtctgtaagattttattttcaagcaaatatttatgacctcaacaaagaagaaccatcttttgttaagttcaccgtagtaacacataaagtaaatgctacctctgatcaaagcaccttgaatggaaggtccgagtctttttagtgttttgcaagggaatgaatccattattctattttagacttttaacttcaccttaaaattagcatctggctaaggcatcattttcacctccatttcttggttttgtattgtttaaaaaaataacatctctttcatctagctccataattgcaagggaagagattagcatgaaaggtaatctgaaacacagtcatgtgtcagctgtagaaaggttgattctcatgcactgcaaatacttccaaagagtcatcatgggggatttttcattcttaggctttcagtggtttgttcctcagttttaaatgtgcaattttcttgctcctatttaagtgttcacaaaaggtaatagtcaatgagctcatcacttcatccatgcaggaagtcaagcattaaaatgtactctttatttctcactggtttctccatactgcaggctccccacatattattttctttttttaactcagctcagaatccttatgccttttgaatcagtgtgataaaatgacagatgtttttgttgattgccagaacagtttaaactttttttaaacaataaatccaataaacataatctcaagacagactcatatgaccatacagaaaagtgtgtcatctatttttcaatcctgtgtagttacttggatgtgaaatattaacaatggcccaaaaatattttcctgaagattgtgtttatataatgtcatcaccaatattttggtctttttgatctctgctaaatgtcaagatttcctttgtaaagtgtcgatcttctaagtagtttctttaagacaattctccgctttaactgattttctttgttgtgaaacacagtagagatttggcaatcaaccattttacttgatctaggtagacagccaagtcagatggcccatgcctagaagctctccattttgaacttttgtcagcattgattaaaagaatcaaataccttgtagttatctatgatgatacaagtaaaaaactaggctgctgacttctgagtattcctgagcctacaattgtaaaattgtagactccattgtaaaatttgtattttttcatcaatctgacaaggcacaaatatgtgccagatatacaaaagcaatgtttctagaaaacagctatcatggatcagaataactgaatttactctcagatctattggctatagttatgtggactcaacccacgtatccagtagatgggaaaaaacaaaagccaaaataagttttttagtgtttccttctgatgaagtttcatgtttgcttgtaataatctccatttctcaaatattatgttccataatagacatacattatgtttaattttttatattttctgacaaaagtaactaaaactaggaccttaaaaagatttagaatgttaaataagtgtactagggtgtatatatttacatatatacactactagagcttccaaaagtaaaatggataattcaaacagaacacaatgtaatatttgtatgtaaataactgaggaggaaaatccatgcttttcatgggctaggatggtttctcccaagagatgacatagtattgcttttgctcatcaggctgtttctcagcaatcattgtttctgcttaataccagctcctagtacgaattatctggcatgttgagagcaactttgtcttcaagtaggacctgatctatctttttccacaaatgtcatgtgtgtgaacaagtttcttccatgtcatctttgagactctacacagaatacacaataaagggagttatttttaaaataagacattctaaagtaaataataaataaggtcattgtcaacgtttttcattcaaaaccattttttaacgtaaatttgctagaaccaccttccaattccaaggcaaggagagacattacaaccctgactcaactggatgggctaaggtttctgataaaatctgaagataaagaaaatggaatattctgcttttttcttccttctaatttcacccttgcctaaggatgagatttcttcccaggttggtatcccagaaatgcagactgtagctatggggcggaagctttgtttctttacctgatcacttgctgtggaaattctagcttattgtgttccaagtagttagtggtttttctccttcagtctccactgttattttgctccttcatccctctttccttgccaatcattagaaaggaaagaagaggaaagagactcgctggagcactggtgagtctctaggaccctgctatcctatcccaacagggctgtcagacggagaactcctaatgtggccatttgaaacacttctcaacattgaaatagacagttgaagttttaaaataacctcttctaagacacggctatgagtaggtaagagagcattcattcccttcaataatatgactgtgttgataaaactgataaccattcacttgcaaatgttattattgaataagtctcacttagctcatttaatattacccaaaagatgctaacaaattctgtttcccacattgtcacagcatgccctttacatctcaggatccaggcaaaagttgaaattcagaaacatagatatgaaatgtaagatacaaagaaaacacctctgcaaagattccgaccacatttatcaaaaagtccccaaagcattcaaaatctttacttaagtcaagtctatttatacgtttaaaagctaaaaacaagatctttttggtaatgcttcaattaaatgttttatctaaatatgtggaaatgataagatgcgtactgcatctctcatataataaagataatcagttttatacagacatattctccatctactggcaattaccaagataagatggcactagaaatttctccagcttacgccatgaatactgcagaagctgatactatccgttgtggttttacaaattctagagggttctagccaaagcaacctaagaataggacatggtagcttaagtttttcagcttcttaactggccacacacacacaagttgtgtttgtacaattcttgaggtcaatcagaaccaaaaaatctgttgctggaagaaatattatcctcttcatagaaatatccaccagcagaaaattggtttctcaaggaatccctactgcccttgtagaaacatcaagattcttgcctggattctcaacataagtctttactcacaggcctattgcttggtttcagaagagtgagaacatgaaagttcataaatgcctgggccactgcaactctaaccactgtgtttcccccagtttgatatggttcaggatacatagtcatagaacagggcatgcagattgtatttaagaccactgcaagtaaggtctaaggcaaaagtaaattaatgagtccaactctggggcatccatttaagagccatttaatccatttaattacaaataatttcaacccatagtcagtgttcttcactgtcttcaaaaatataaaaagtacaaggaattgtgtacattacaaactgctccagaaacaaaaccaaatgtggatagctttgtgagctgcagtgtgtggcaaatgttcaaccttttgttatgcaatatcacccatatcaaataccattcttaaagcagtagacagatgagtcaagttcaatttaatgcaaacaatattactgtgttctaagcgcttctgttactcgaaaggggtctgatccagaccccaaaagagggttcttggacctcatgcaagaaagaattcaggagtaaagtgaaagtgaaatgggtcaggatcaatggtctcattgtctaaggtgttatccgagctcgttgtctcacaaccaagaaaattaaggagcatggacacaaagggtgaggttggagcaaaagtttaataagcaaaagaggaaagctctctgcagcagagacgggagcccaagtgggttgctgtttttacagctgaatccaaaagcttttataagaaactcctctcatctctgcagctatttgagtaacttctcttatctgaaaagctgtctgtacaactgcctctatctatgcagctatggggtgtctctaggcgagcacaaagcatagcttctcttgttgtataattgtgggtttgttttaagtaagccactttcctccctgcaagttcccacggagcagaaaaaaggaggaaactttttcctgggagcccactaatcacacagtgaacaaaaggcttctatgctgggccttactttctaacagtgcagcagttatagtctgagttttctccaggctgctccatttttgcctgtagctatgatttttcaggcagcctgcttctccgaggactagtcttagctgtttacctaactgattggtccttttcttctccctgaaaagcaagtttattaagaaagcaaaggaataaagaatggctactccataggcagcgtagccccaagggctgctggttggctatttttgtggttatttcttgattatatgctaaacaaggggtggattattcatgggttttctgggaaaggagtgggcaattcccagaactgagggttcttccccttttaagaccgcatagggcaacttcctgacattgccatggcatttgtaaactgtcgtggtactggtgggaatgttttttagcatgctaatgcaatataattagtgtataatgagtagtgaggacaaccagaggtaactttcattgccatcttggttttagtggggtttggccggcttctttaccacatccttttatcagtaagatcttcgtgacctgtaccttgtgccaacctcctatctcttcctgtgacttggaatgcctaacctcctgggaatgcagcccagtaggtctcagccttattttacccagcctctattcaagatggagtcgctctggttcaaacacctctgacacttgaattacaaatataaggaccattgacactgagattttaagggaggaaaaacagattgacagtggactaaaggtacttttgtagcaaggtactttccacacaatattgaataaatgcagtgtatacatttttaaaggaattttaagagctcggaaatcattaaaattaagtttatcaatttttgaaagcatcttctgactcaaatatatgaaaagattattctagaccttaggcagatattcagagaaagatcagttttctatgggttttttccatcatgcattttatacaaatttaatatactttttcaactcactttgcatttcctgttaccttgtactgagaacttcattaaaacctgcacttgaaattgcacttaaactttacaaaatcacagaagaagttgttttccataggtgtggggtgggatgttagagctctagacattaattcctaggaatcgcatacctacggaaaacagtcctctgacctcctgtgacacatggggtgagcccttcctttttgtctcaatctcaaaaaacagtaagtctttaatccatttgcatcaaaaagtacttcatatgctcgaaactaaagtaaaactttattgaaatacattaatatgctcctggaacatactactgggtctcagacagtgcagaagctttattgtttatttgggaagagcaaggtaagaatcaagtagaaatgataaagggcaaggaaaaaagatgaaagcttactcatattaaccattctaccattggaattatttgccaacacaccttgctgctacttagagaaagtagttcacctcactaccttttatccaaagaaattatctctaaaagcactcagacattttgtagagcaagtagcaatctattcaaagttgtaaaggttctgtagaatctctcagaccaggtacaggacctaccaaaggccacagccaagcacaagcagctacattagacagttttacagctctgtatttcaggaaaacttctgtcctgtgggagcaatacaaaactataccagttttttgttagtgtaaaaattgcccaatattaaccagagcagcccaaaatatttcagggtaagaatagatatatttatatttttcagatgatatatcttcattttcgattttgaaagaacagtaatactaattatatcccatgtaaggggctactgacaattttgatggtacctgaatttgcctctatcatgcatctcaatgatttgttgtcatccaaagctatttcatgaatcaaatatcgttttctacctgccccacaactgtgtacataaaacctaaacctctgaagcaataaacctcttccattacacaggtttagattcagagttttcttgcttaagttccaactaaaagtattacattcttagcataagtatactcataaagaaaaataagtatttgttttaggttttagagagagagcacagagtccctttgagacagtggggaaaattcatcttcatattgtcacatgcactgtaataggaatgtttagcaaaaaaaaccttccagagaaaggtggtttccaatattacctacaacttcctttgcaatttgatttttgaaaggacctaaaagttgaaaacaggctatcacatcccatttgctttaaagtctcttaaacttacgctctttcgcttcaaatgcataaatgttttatttaagtttgcattgcccactaaggctagacatttttttttttttttttttttttttttttttgagacagagtctcgctctgtcgcccaggctggagtacagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatgatctcgatctcctgacctcgtgatccgcccgcctcggcctcccaaagtgctgggattacaggcgtgagccaccgcgcccggcccggctagacattttttgataaattcacagggttacaaaataccaaacggaaatgagataagtggtataaaccacagaagatataggagaagagaaaaaaaaaagaggaaataaagaagacaactcttttcctaagagtctgggtaaaattgaacatagccatattcactgaacaacatgagtgagcttcattaatttaagcacagcaaaactgctttaattaacaagaccagagagaagggagaggagactacatttgtgtgacctaatggttgtgatttcactgtccaagaggacaaagacaaagaaattctgggaaggagaacaacaattatattcccccatttcaagaagggcagaagtgtcccaacactacccaatatttgcaaaattcaaatgtctcataggctcttcttccctggttccctcaggagctgggtttctgggttgcagaagtgcttttcatattctgtatctggttgtggtggcaatgtcaccaccctacactgctgtgacaccgaaacaaccaagcctagaatcagctggtgcctcttttcatctgcagggtagatttggcttccatggttgttcactgctctgtgttaggaaggctcagtgacaggtgtacagccttcagtaatgcctcaaaggttctccaagcagaggtaaacatgtgggtcctgctggtgacatattagacttcttactttccccaaataaaaaagtgcctgctgggcgcggtggctcacgcctgtaattccagcactttgggaggccgaggcgggcggaacacaaggtcaggagatcaagaccatcctggccaatatggtaaaaccccatctctactaaaaatacaaaattaggaaggcgtggtggtgcacgcctgtaatcccagctagtcgggaggctgaggcaggagaattgcttgaactggggaggcggaagttgcagtgagccaagatcgcagcattgcactccagcctgggcaacagaatgagattgtctcaaaaaaaaaaaaaagtgccacatgccatgctatgtgcccaaagtttccttcacacaacacagccttgagatgcagtattaaattctacacttttcctaccatagtgatacatgtggcttttctttgctgtgttctgagatgtcatgctttgaaatcagtggccattatcatctaaggattccgccagagacttccaaaagaagaggtctcatttataaagtgaatttgaataaaatgaccagttaggtgttttcagaaacacctatgccctacttgcctactcttcaagggtttaggggcttagggggaggttttgtttgggttttttgttgctgttgcttgtttatttgtttgttgtgtttaagacgttttacttgtccctgaaatgtttgtcatcacacagatacacgctcaggataagaactaccagactagattaggaggtccacaccaccaattgagatgtacctgtgctcatgacttgacattgtggtgggccggctacaaccctccccacccctcgctttcactaaataacaactctcttctctccatcattttgacttagagccagtcagaattcaatctccaatatcctgactagcacaagaaatccataggttgattcttgttctcctgcatctctgcaggtggcaaacctgattcctaatgcctgttcctgcctctgcaggggttcattcagaaaacaggaaaataacaaaggcttcctgtaattctctttggctgtaatacaatttgttcccgtctgcccccaggctcacccagtgctctgtctcagtggtaagctgtaactgatcactgctgtattaactcaaaactcatttgctttatggaaattcatggcccttatttctcaagggaccagagaaaaccaataggcctactccccagctgagtactttccatgcaagctaccgcatctgtataaattaggttcaaataacagagtatttccaggatttataaattcagtattacaaatagtaatctggcaagtgttctcaggatccccttgctacctgtaattcaatcatataacttctgaatgggctgggggaaaataacaataagaaaaactggtgtttacctgaagatctgcccagtgatttgtgtgttttcttaataaactttacccacttattaaaagaataaaatgaaggtggagttaattctgactacgggattcctttttcacttttataatgaactccttccttctaactaaatcttatcataagcaaatctatgcaccaaattatttagtacaattcctaataacagctgaaggaccatttatttgaagcaatgttcaccatagcaaaattccagtgaagtctaagaactgggacagtccgttgaggatccttgtgccaggatgtatgttgccccatgaatgtgcacatgcatattaaaatatgggcacctcttttaattcttttttttctcataataagtttgaaactcacagtaggaaattgagagatcaatttggttactgttttatcattgatcctgaagacagttgaagcaatcatactggttgttctcgaactagctggtttcccagagacagctggagactgagcacataaagacatcattgaggaaaaaggctaccttgtacctcatggagagctgaaggtctgataaatgggaactgccaggtaatagctatgctatttctgacataaatttaaaaactagtattgtttcttctagctctgtttttgcatagtgcacagagatctttgtaaaaaacaggaaattaatgttaaattggatctataaacataagtcaatttggctctattatgtcaaaagagaataggagttttaacttatatctgtgttttattaatattttgaagtataggaacctcatggtgtagcaggatgagccacagacaaaacctctcagacaccgagttgtagaaggaagggctttattcagctgggagcatcgaccagctactgtctcaaaatccaagctccctgagtacacaatttctgtcccttttaagggctcacaacactagatttcacatgaaagggtcgtgattgatttgagcaagcaaggggtatgtgacaggggctgcatgcaccggtggtctgggaggaacagaacaggacagggagttcttctatacaatagagaacagaacaatgttcttctatacaatgtaaggaatctatgaataacatcggcttctaaatcataagttgatttttaactactgggtttaggccaggcgggcccaggcctggtttcgggcctggcgctgagctgcctgtatttggttttacttccttgttgtttttactgaatatgaaacaatataaaacaatgtgagagggtctttctctcctctcaatgtcaacatcatatatgattggagacttccacataattgagttttagtgcccactgttacagaaaatcataatggaaaaactaaaatgtcaataataatttcagatgtgtattttagttctcataagaacatctacattcatttgaaaaatagttctatatctattcttgaaacatatttctttagttcaaggtctgatgagctcccagactgttggggattgctctacagctgctcaagcttttgaagactcctgtgatttttttaaatggctggtttggttgaagtttctcttatcagtcaggcactttgcattttaagcgtactttaccaccgacaccctccccccccagcacacacacacacacacacacacacacacacacaacatagtgaaatggacccgtgggaattatatgatagttgtaatcaaaataaaatgcaatcaatactaaaatacaatttaccaaaggcttacctccttatttgaaactccagcatcaataatttacttgcactcttgttatttacatttgtactctggaagtaaacttaaaatgaaaattagaatttgctttcaattatactatctctatctaaatcttaatttgaaatttaaattattttgtctctacccaaaccatcgatttcatggaaatgtttaaattttcttttttttttttttttttgatggagtctcactctgtcgcccaggctggagtgcagtggctcaatcttggctcactgcaacctctgcctcccgggttcacaccattctcctgcttcagcctcctgagtagctgggactacaggtgcccgccacaacacctggctaattttttgtatttttagtagagatggggtttcaccacgttagccaggatggtttcgatctcctgacctcgtgatctgcctgcctcggcctcccaaagtgttgggattacaggtgtgagccactgcgcctggccagaaatgtttaaaatttcataaatcttgaatccataaaattcaatgctaccatttataatttaatactcctaccataaaaatttctgtgttaggcatagctatatgaatatttgcctataaatccccatcaatttaataggaattaagttagaaatactagtatatatattccctttatatactaattgtatatccatataaaagcattagtaccattatatgaaagtatatatgccattccataaaaatatatctaccaatataaatagaatatataaagggaatatatatataccaatataaatgggaatttatattggcacatacatttccattattttaatgggaattaaagaaaaaatgcctgttttcactaagtcatccttcccctggcaatacatttcctgaacttttacatacttaaatagccagttatgaaaatgtaaaacaatgagtgatcttgttgttttcattttattatgttatatgaaaaaaagaaaccttgtaagtgcagatttattttaaaaaattgaaaaactactctgtcaaacatagcaaataggaaagttaaaaaaaagtaagtctaaagattaggtgctctgtcacttgtcattacatttttttataacattaatgacacattttttgctatccaaatataatttccactagaaaagaaaagatgtcctccatgttttaaattgcatttgaaattcaggtaactaataacccttggttttaaaagatacatataatgtttccgtaggaaaaaaatcaaaatattgtaaagaatctgagtaaatgagagcctctcatggttgattaagttcagacattttaaacagactcctgcccacaaactatttttcctctccaggaataagaatggcaactgaattgttccttctttattctatagctttaagtcaaacctaacataagcaatcaacccttccacccattgtcctctttctagctgcttatattcctgagtctgaaagaagggcagaattaattcgtttccttgacagtgttgggggtgaaataaaagatagacccctgctgctctgcacgtagattcagtttgtatgccagggtgacattttaatttacagtagtccagacacctaaacaggacatagaaatgtcaactggtcatattaaaataaaaaagtatagaatagtttctgttttacggattttgtatccaagtaaatgaaaacacaatcacctttcaaaacatggtatgaaatccacagttgtaacagtgagcacaacatgctttctgtgtgtgtttttaagctctttccccacatctgcttttcaaaaatatttgcctggaaacctgtatttcgcctatagagacaaatacatatattgtttgttgtgataatgcacaaaaaggaatgttaaaaaaaaacacacaacattgttttccttttgatggtctgggagtttttctataagtttttggtttttttttttttttctcttcattagtgtgttagttccatcatcatgtctgtttactattgaaaaataaatatcttcatttaaagtagagacaaagctactatttcacatttccaggtaggacaggatgatcagatgcagctttcaaaagaactacctgctaaatcataatggtctcatgtgcagatcaaaatgtcaactagtatatcaaacagtgagcaattcactaaattatttttacattactcctatttgactgtttcagatagttgtatataaaaaatataaatttttgcattgtattgttcttgatgtaccagcatacataaacatattaggctgtattgtaagtttcctgcatgtaatatgtaatgtgtaattatatgtgataaataaaacctaaaactgataca

SEQ ID NO: 12 - protein, 400 aa, Homo sapiens
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPVKALDFRTPRNAKIINVCNWILSSAIGLPVMFMATTKYRQGSIDCTLTFSHPTWYWENLLKICVFIFAFIMPVLIITVCYGLMILRLKSVRMLSGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP